SEQUENCE LISTING (51 sequences)

SEQ ID NO: 1 - protein, 227 aa, synthetic construct
  REGION 1..227: note = Synthetic
AYALELALGALRLEDRARELIKEAEKKGDPEKLREALEALEEAVRLVEEAIKLRPDMDLAVEIAVRLARMLKRVAELLQELAKKTGDPELLKLALRALEVAVRAVELAIKSNPDNDEAVETAVRLARELKKVAEELQERAKKTGDPELLKLALRALEVAVRAVELAIKSNPDNEEAVETAKRLAEELRKVAELLEERAKETGDPELQELAKRAKEVADRARELAKKS

SEQ ID NO: 2 - protein, 125 aa, synthetic construct
  REGION 1..125: note = Synthetic
NDEKEKLKELLKRAEELAKSPDPEDLKEAVRLAEEVVRERPGSNLAKKALEIILRAAEELAKLPDPEALKEAVKAAEKVVREQPGSNLAKKALEIILRAAAALANLPDPESRKEADKAADKVRRE

SEQ ID NO: 3 - protein, 119 aa, synthetic construct
  REGION 1..119: note = Synthetic
ELAKRADDKDVREIVRDALELASRSTNDEVIRLALEAAVLAARSTDSDVLEIVKDALELAKQSTNEEVIKLALKAAVLAAKSTDEEVLEEVKEALRRAKESTDEEEIKEELRKAVEEAE

SEQ ID NO: 4 - protein, 174 aa, synthetic construct
  REGION 1..174: note = Synthetic
DERQKQREEVRKLAEELASKATDEELIKEIKKCAQLAEELASRSTNDELIKQILEVAKLAFELASKATDEELIKRILKCCQLAFELASRSTNDELIKQILEVAKLAFELASKATDEELIKLILACCVLAFELASRITNDEEIKQILEFAKEAFERASKATDEEEIRKILAKCIA

SEQ ID NO: 5 - protein, 405 aa, synthetic construct
  REGION 1..405: note = Synthetic
  REGION 1..2: note = MISC_FEATURE - optionally absent
  REGION 398..405: note = MISC_FEATURE - optionally absent
MGDRSEHAKKLKTFLENLRRHLDRLDKHIKQLRDILSENPEDERVKDVIDLSERSVRIVKTVIKIFEDSVRELEKAILWLAEELAKSPDPEDLKRAVELARAVIEANPGSNLSRKAMEIIERAARELSKLPDPEAQRTAIEAASQLATMAAATGNTDQVRRAAELMVEIARLAGTEEAQDLALDALLDVLETALQIATKIIDDANKLLEKLRRSERKDPKVVETYVELLKRHEEAVRLLLEVARVHEELVRFTIIEEKVRSPDCEDIRDAVREAEELLRENPSEMAEELLRRAIEAAVRCPDCEAIREAVRAAEELLRENPSTEAEELLRRAIEAAVRCPDCEAIREAVRAAEELLRENPSEEAKELLRRAIESAKKCPDPEAQREAKRAEEELRKEGSHHHHHH

SEQ ID NO: 6 - protein, 185 aa, synthetic construct
  REGION 1..185: note = Synthetic
  REGION 178..185: note = MISC_FEATURE - optionally absent
DEEDESYELVEHIAEELEEIAEEIAEAVENLAQAIIEALYVAWESNQQINEQVQEVEQSMAELAYLLGELAYKLGEYRIAIRAYRIALKHDPNNAEAWYNLGNAYYKQGDYDEAIEYYQKALELDPNNAEAWYNLGNAYYKQGDYDEAIEYYQKALELDPNNAEAKQNLGNAKQKQGGSHHHHHH

SEQ ID NO: 7 - protein, 271 aa, synthetic construct
  REGION 1..271: note = Synthetic
  REGION 1..2: note = MISC_FEATURE - optionally absent
  REGION 264..271: note = MISC_FEATURE - optionally absent
MGDIEEAKEESRKIADHGHDGHKAVADLQRLNIELAHKLLDEVEQLQNLNDELARELLDLVDRLAELLIDLVRKTSELTDEDTIRREILKVDVRMLAISLAASAKDEELRKEIKKCLQLAEELASRSTNKELQKQAMEVAKLALELARKATDEELIKEILKCCQLAFELASRSTNDELIKQILEVAKLAFELASKATDEELIKEILKCCQLAFELASRSTNDEEIKQILETAKEAFERASKATDEEEIKEILKKCQEKFEKKSGSHHHHHH

SEQ ID NO: 8 - protein, 350 aa, synthetic construct
  REGION 1..350: note = Synthetic
  REGION 1..2: note = MISC_FEATURE - optionally absent
  REGION 343..350: note = MISC_FEATURE - optionally absent
MGVEELLLLARAAHHSGTTVEEAYKLAKKLGISVKELLLLARAAHNSGTTVEEAYKLALKLGISVEELLLLAKAAHYSGTTVEEAYKLALELGISVRELLLLAKAAHFAGRTVREAYALELALGALRLEDRARELIKEAEKKGDPEKLREALEALEEAVRLVEEAIKLRPDMDLAVEIAVRLARMLKRVAELLQELAKKTGDPELLKLALRALEVAVRAVELAIKSNPDNDEAVETAVRLARELKKVAEELQERAKKTGDPELLKLALRALEVAVRAVELAIKSNPDNEEAVETAKRLAEELRKVAELLEERAKETGDPELQELAKRAKEVADRARELAKKSGSHHHHHH

SEQ ID NO: 9 - protein, 187 aa, synthetic construct
  REGION 1..187: note = Synthetic
  REGION 1..2: note = MISC_FEATURE - optionally absent
  REGION 180..187: note = MISC_FEATURE - optionally absent
MGDEEDESYELVEHIAEELEEIAEEIAEAVENLAQAIIEALYVAWESNQQINEQVQEVEQSMAELAYLLGELAYKLGEYRIAIRAYRIALKHDPNNAEAWYNLGNAYYKQGDYDEAIEYYQKALELDPNNAEAWYNLGNAYYKQGDYDEAIEYYQKALELDPNNAEAKQNLGNAKQKQGGSHHHHHH

SEQ ID NO: 10 - protein, 478 aa, synthetic construct
  REGION 1..478: note = Synthetic
  REGION 1..2: note = MISC_FEATURE - optionally absent
  REGION 471..478: note = MISC_FEATURE - optionally absent
MGQRSGHARQLKRHLHRLRRHLERLDKHAKHLRQILSERPQDERVKDSIDLLEESVRIVKISIKIFEASVRALLWAINKEAEELAKSPDPHDLHRAVRLARAVVQADPGSNLSKKALEIILRAAAELAKLPDPNALAAAARAASQVQREQPGSNLAKAAQEIMRQASRAAEEAARRAKETLEKAEKRGDPKTALQAVRTVVKVAAALNQIATMAGSEEAQERAARVAAEAAELALRVFELAEKQGDPHVARRARKLIQTVLQILLRILTQILETATKIIEEANKLLRKHRRSSRKDPKLVETHVELVKRHERLVRQHLKIALMHALAVLELAFPDAEAAKLASKAAKEAEELCKQSTDERLCDLLAELAALLIELAARYPDSEAAKLALKAALEAIELCKQSTDEELCEELVKLAQKLIELAKRYPDSEEAKRALKEAKELIEQCKESTDEDECRELVKRAEELIREAKEGSHHHHHH

SEQ ID NO: 11 - protein, 486 aa, synthetic construct
  REGION 1..486: note = Synthetic
  REGION 1..9: note = MISC_FEATURE - optionally absent
MHHHHHHGSSDEEEKKELEKRIEEAAQRAREAAERTGDPRVRELARELARLAERARELVERDPSSSDVNEALKLIVEAIEAAVRALEAAERAGDPELREDAREAVRLAVEAAEEVQRNPSSSTANLLLKAIVALAEALAAAANGDKEKFKKAAESALEIAKRVVEVASKEGDPEAVLEAAKVALRVAELAAKNGDKEVFKKAAESALEVAKRLVEVASKEGDPELVLEAAKVALRVAELAAKNGDKEVFQKAAASAVEVALRLTEVASKEGDSELETEAAKVITRVRELASKQGDAAVAILAETAEVKLEIEESKKRPQSESAKNLILIMQLLINQIRLLVLQIRMLDEQRQNQQREARVKSNEMERLAEVLRLSARARRGAMSGSEEDQERLRKEMEEERKHMEEVEKELRKVEEKMKSHEDTSLRLLVLIARLLINQIRLLILQIRSLSNLERNQAREAMVESNEMEREAETLRLSAREQRRAG

SEQ ID NO: 12 - protein, 478 aa, synthetic construct
  REGION 1..478: note = Synthetic
  REGION 1..2: note = MISC_FEATURE - optionally absent
  REGION 471..478: note = MISC_FEATURE - optionally absent
MGDRSGHAKKLKTHLENLRRHLDRLDKHAKQLRDILSEHPHDERVKDSIDLLEESVRIVKISIKIFEASVRALLWAINKEAEELAKSPDPEDLKRAVELAEAVVRADPGSNLSKKALEIILRAAAELAKLPDPDALAAAARAASKVQQEMPGSNLAKAAQEIMRQASRAAEEAARRAKETLEKAEKDGDPETALKAVETVVKVARALNQIATAAGSEEAQERAARVAAEAAELALRVFELAEKQGDPEVARRARELIEKVLDLLLSLLTQILQTATKVIDDSNKLLEKLRRSHHHDPKLVETHVELVKRHERLVRQHLKIALMHALAVLELAFPDAEAAKLASKAAKEAEELCKQSTDERLCDLLAELAALLIELAARYPDSEAAKLALKAALEAIELCKQSTDEELCEELVKLAQKLIELAKRYPDSEEAKRALKEAKELIEQCKESTDEDECRELVKRAEELIREAKEGSHHHHHH

SEQ ID NO: 13 - protein, 486 aa, synthetic construct
  REGION 1..486: note = Synthetic
  REGION 1..9: note = MISC_FEATURE - optionally absent
MHHHHHHGSSDEEERKELEKRIREAAQRAREAAERTGDPRVRELARELARLAERARELVERDPSSSDVNEALKLIVEAIEAAVRALEAAERAGDPELREDAREAVRLAVEAAEEVQRNPSSSTANLLLKAIVALAEALAAAANGDKEKFKKAAESALEIAKRVVEVASKEGDPEAVLEAAKVALRVAELAAKNGDKEVFKKAAESALEVAKRLVEVASKEGDPELVLEAAKVALRVAELAAKNGDKEVFQKAAASAVEVALRLTEVASKEGDSELETEAAKVITRVRELASKQGDAAVAILAETAEVKLEIEESKKRPQSESAKNLILIMQLLINQIRLLVLQIRMLDEQRQRLEQQMRMEVRQLEIRSECLRKESAVVSMVNSVGTHDQMKLKEQMEEEERHTEKVEKEIRKVEEKMKSHEDTSLRLLVLIARLLINQIRLLILQIRSLSNLELRLQQQMRMEVEQLRIRSQCLQEESEVVEEVE

SEQ ID NO: 14 - protein, 478 aa, synthetic construct
  REGION 1..478: note = Synthetic
  REGION 1..2: note = MISC_FEATURE - optionally absent
  REGION 471..478: note = MISC_FEATURE - optionally absent
MGNRSLHANKLKTHLENLREHLKRLDEHAKQLRDILSEHPHDERVKDSIDLLEESVRIVKISIKIFEASVRALLWAINKEAEELAKSPDPADLERAVRLAAAVVRADPGSNLSKKALEIILRAAAELAKLPDPKALAAAAEAASRVQREQPGSNLAKAAQEIMRQASRAAEEAARRAKETLEKAEKDGDPRTALQAVMTVVEVAKALNIIATMAGSEEAQERAARVAAEAAELALRVFELAEKQGDPEVAHNARKLIEIVLHILLQILTQILETATKIIREANELLEKHRRSHHHDPKLVETHVELVKRHERLVRQHLKIALMHALAVLELAFPDAEAAKLASKAAKEAEELCKQSTDERLCDLLAELAALLIELAARYPDSEAAKLALKAALEAIELCKQSTDEELCEELVKLAQKLIELAKRYPDSEEAKRALKEAKELIEQCKESTDEDECRELVKRAEELIREAKEGSHHHHHH

SEQ ID NO: 15 - protein, 408 aa, synthetic construct
  REGION 1..408: note = Synthetic
  REGION 1..9: note = MISC_FEATURE - optionally absent
MHHHHHHGSSEEEQERIRRILKEARKSGTEESLRQAIEDVAQLAKKSQDSEVLEEAIRVILRIAKESGSEEALRQAIRAVAEIAKEAQDSEVLEEAVRVIEEIAKESGSEEALRQAKRAIEEIAREARDLRVEALALLAMARLYLLMVKLEQEEKAKEFQELLKELSERSEELIRELEEKGAASEAELARMKQQHMTAYLEAQLTAWEIESKSKIALLELQQNQLNLELRLMKEILRRKEKALELRKLLLAAQALVQAAAQAERQTREDDSLREAEELLRRSREYLKKVKEEQERKAKEFQELLKELSERSEELIRELEEKGAASEAELARMKQQHMTAYLEAQLTAWEIESKSKIALLELQQNQLNLELRLHEAQKRRKEKALELRKLLLAAQALVQAAAQAERQTR

SEQ ID NO: 16 - skipped

SEQ ID NO: 17 - protein, 324 aa, synthetic construct
  REGION 1..324: note = Synthetic
  REGION 1..2: note = MISC_FEATURE - optionally absent
  REGION 317..324: note = MISC_FEATURE - optionally absent
MGCDAIQAAAALGEAGISSNEILELLAAAAELGLDPDAIQAAAQLGEAGISSEEILELLRAAHELGLDPDAIAAAADLGQAGISSEEILELLRAAHELGLDPDAIQAAAALGEAGISSEEILELLRAAHELGLDPDAIQAAAQLGEAGISSEEILELLRAAHELGLDPDCIAAAADLGQAGISSSEITALLLAAAAIELAKRADDKDVREIVRDALELASRSTNDEVIRLALEAAVLAARSTDSDVLEIVKDALELAKQSTNEEVIKLALKAAVLAAKSTDEEVLEEVKEALRRAKESTDEEEIKEELRKAVEEAEGSHHHHHH

SEQ ID NO: 18 - protein, 99 aa, synthetic construct
  REGION 1..99: note = Synthetic
  REGION 1..2: note = MISC_FEATURE - optionally absent
MGSIEEAEEESRKIADKGSDGHKAVADLQRLNIKLAEDLLRHVEELQELNIDLARQLLRLVEELQKLNIDLVRKTSELTDEKTIREEIRKVKEKSKEIV

SEQ ID NO: 19 - protein, 382 aa, synthetic construct
  REGION 1..382: note = Synthetic
  REGION 1..2: note = MISC_FEATURE - optionally absent
  REGION 375..382: note = MISC_FEATURE - optionally absent
MGVEELLLLARAAHYSGTTVEEAYKLALKLGISVEELLLLARAAHQSGTTVEEAYKLALKLGISVKELLLLAQAARNSGTTVEEAYKLALKLGISVEELLLLAKAADFSGTTVEEAYKLALKLGISVEELLLLARAAHYSGTTVEEAYKLALKLGISVEELLLLARAAHQSGTTVEEAYKLALKLGISVKELLLLAQAARNSGTTVEEAYKLALKLGISVEELLLLAKAADFSGTTVEEAYKLALKLGISVEELLLLARAAHYSGTTVEEAYKLALKLGISVEELLLLARAAHQSGTTVEEAYKLALKLGISVKELLLLAQAARNSGTTVEEAYKLALKLGISVEELLLLAKAADFSGTTVEEAYKLALKLGISGSHHHHHH

SEQ ID NO: 20 - protein, 106 aa, synthetic construct
  REGION 1..106: note = Synthetic
  REGION 1..2: note = MISC_FEATURE - optionally absent
MGDIEEAKEESRKIADHGHDGHKAVADLQRLNIELAHKLLDEVEQLQNLNIELARDLLRLVEELQRLNIDLVRKTSELTDEKTIREEIRKVKEESKRIVEEAEEEI

SEQ ID NO: 21 - protein, 382 aa, synthetic construct
  REGION 1..382: note = Synthetic
  REGION 1..2: note = MISC_FEATURE - optionally absent
  REGION 375..382: note = MISC_FEATURE - optionally absent
MGVEELLLLARAAHHSGTTVEEAYKLALKLGISVKELLLLARAAHNSGTTVEEAYKLALKLGISVEELLLLAKAAHYSGTTVEEAYKLALKLGISVRELLLLAKAAHFSGTTVEEAYKLALKLGISVEELLLLARAAHHSGTTVEEAYKLALKLGISVKELLLLARAAHNSGTTVEEAYKLALKLGISVEELLLLAKAAHYSGTTVEEAYKLALKLGISVRELLLLAKAAHFSGTTVEEAYKLALKLGISVEELLLLARAAHHSGTTVEEAYKLALKLGISVKELLLLARAAHNSGTTVEEAYKLALKLGISVEELLLLAKAAHYSGTTVEEAYKLALKLGISVRELLLLAKAAHFSGTTVEEAYKLALKLGISGSHHHHHH

SEQ ID NO: 22 - protein, 128 aa, synthetic construct
  REGION 1..128: note = Synthetic
  REGION 1..8: note = MISC_FEATURE - optionally absent
HHHHHHMGTEDLKYSLERLREILERLEENPSEKQIVEAIRAIVENNAQIVEAIRAIVDILRLIVSNNAAIVAILALIVDNNRAIVEILALIVENNRAIIEALEAIGGGTKILEEMKKQLKDLKRALET

SEQ ID NO: 23 - protein, 103 aa, synthetic construct
  REGION 1..103: note = Synthetic
  REGION 1..2: note = MISC_FEATURE - optionally absent
  REGION 96..103: note = MISC_FEATURE - optionally absent
MGVEELLMLAIAAAASGTTVEEAYKLALKLGISVTELLALAAAAAASGTTVEEAYKLALKLGISVEELLMLAQAAAFSGTTVEEAYKLALKLGISGSHHHHHH

SEQ ID NO: 24 - protein, 324 aa, synthetic construct; identical to SEQ ID NO: 17
  REGION 1..324: note = Synthetic
  REGION 1..2: note = MISC_FEATURE - optionally absent
  REGION 317..324: note = MISC_FEATURE - optionally absent

SEQ ID NO: 25 - protein, 137 aa, synthetic construct
  REGION 1..137: note = Synthetic
  REGION 1..2: note = MISC_FEATURE - optionally absent
MGSAEELLRRSREYLKKVKEEQERKAKEFQELLKELSERSEELIRELEEKGAASEAELARMKQQHMTAYLEAQLTAWEIESKSKIALLELQQNQLNLELRLHEAQKRRKEKALELRKLLLAAEALVEAARQAERETR

SEQ ID NO: 26 - protein, 187 aa, synthetic construct
  REGION 1..187: note = Synthetic
  REGION 1..2: note = MISC_FEATURE - optionally absent
  REGION 180..187: note = MISC_FEATURE - optionally absent
MGSEEEESKRLVEEIAKRLKKIAEEIARAVEKLARAIIEALEVAWRSNKKINEQVQRVEQSMAELAYLLGELAYKLGEYRIAIRAYRIALKHDPNNAEAWYNLGNAYYKQGDYDEAIEYYQKALELDPNNAEAWYNLGNAYYKQGDYDEAIEYYQKALELDPNNAEAKQNLGNAKQKQGGSHHHHHH

SEQ ID NO: 27 - protein, 165 aa, synthetic construct
  REGION 1..165: note = Synthetic
  REGION 1..2: note = MISC_FEATURE - optionally absent
  REGION 158..165: note = MISC_FEATURE - optionally absent
MGTLVEILARAQIESSRVNIELAREALERAKHLHREAKGLAEKMYKAGNAMYRKGQYTIAIIAYTLALLSDPNNAEAWYNLGNAAYKKGEYDEAIEAYQKALELDPNNAEAWYNLGNAYYKQGDYDEAIEYYQKALELDPNNAEAKQNLGNAKQKQGGSHHHHHH

SEQ ID NO: 28 - protein, 271 aa, synthetic construct; identical to SEQ ID NO: 7
  REGION 1..271: note = Synthetic
  REGION 1..2: note = MISC_FEATURE - optionally absent
  REGION 264..271: note = MISC_FEATURE - optionally absent

SEQ ID NO: 29 - protein, 405 aa, synthetic construct; identical to SEQ ID NO: 5
  REGION 1..405: note = Synthetic
  REGION 1..2: note = MISC_FEATURE - optionally absent
  REGION 398..405: note = MISC_FEATURE - optionally absent

SEQ ID NO: 30 - protein, 486 aa, synthetic construct
  REGION 1..486: note = Synthetic
  REGION 1..9: note = MISC_FEATURE - optionally absent
MHHHHHHGSSDEEEKKELEKRIEEAAQRAREAAERTGDPRVRELARELARLAERARELVERDPSSSDVNEALKLIVEAIEAAVRALEAAERAGDPELREDAREAVRLAVEAAEEVQRNPSSSTANLLLKAIVALAEALAAAANGDKEKFKKAAESALEIAKRVVEVASKEGDPEAVLEAAKVALRVAELAAKNGDKEVFKKAAESALEVAKRLVEVASKEGDPELVLEAAKVALRVAELAAKNGDKEVFQKAAASAVEVALRLTEVASKEGDSELETEAAKVITRVRELASKQGDAAVAILAETAEVKLEIEESKKRPQSESAKNLILIMQLLINQIRLLVLQIRMLDEQRQNQQREARVKSNEMERLAESLRLSARDRRGAMSGSEEDQERIRKRMEEEEKDAEKVEKELRKVEEKMKSHEDTSLRLLVLIARLLINQIRLLILQIRSLSNLERNQAREAMVHSNEMERRAEVLRLSAREQRRAG

SEQ ID NO: 31 - protein, 188 aa, synthetic construct
  REGION 1..188: note = Synthetic
  REGION 1..9: note = MISC_FEATURE - optionally absent
MHHHHHHGSTEDEIRKLRKLLEEAEKKLKKLEDKTRRSEEISKTDDDPKAQSLQLIAESLMLIAESLLIIAISLLLSSAGSTGAEDEIRKLRKLLEEAEKKLKKLEDKTRRSEEISKTDDDPKAQSLQLIAESLMLIAESLLIIAISLLLLAEQAAREARIKERVKHAAEKMVRAAEAQAEFARLRAQ

SEQ ID NO: 32 - protein, 408 aa, synthetic construct; identical to SEQ ID NO: 15
  REGION 1..408: note = Synthetic
  REGION 1..9: note = MISC_FEATURE - optionally absent

SEQ ID NO: 33 - protein, 60 aa, synthetic construct
  REGION 1..60: note = Synthetic
  REGION 1..2: note = MISC_FEATURE - optionally absent
  REGION 53..60: note = MISC_FEATURE - optionally absent
MGDKAERSLDKQRRVAEELQKIIEKLQRAVKELQDVLETLKKVSTEQDRTTKGSHHHHHH

SEQ ID NO: 34 - protein, 286 aa, synthetic construct
  REGION 1..286: note = Synthetic
  REGION 1..9: note = MISC_FEATURE - optionally absent
MHHHHHHGSEREELSELAERILQKARKLSEEARERGDLKELALALILEALAVLLLAIAALLRGNSEEAERASEKAQRVLEEARKVSEEAREQGDDEVLALALIAIALAVLALALVACSRGNSEEAERASEKAQRVLEEARKVSEEAREQGDDEVLALALIAIALAVLALAIVASCRGNKEEAERAAEDAIKVAMEALEVLLSAVEQGDLKVALAAVIAILLAIAALLMVIIKRRQDEKMERSLDKQRRVAEELQKIIEKLQRAVKELQDVLETLKKVSTEQDRTTK

SEQ ID NO: 35 - protein, 50 aa, synthetic construct
  REGION 1..50: note = Synthetic
  REGION 1..2: note = MISC_FEATURE - optionally absent
  REGION 43..50: note = MISC_FEATURE - optionally absent
MGTEDELKERQDRLIEKFIKAMAKAASAHAELMRINSELVSRGSHHHHHH

SEQ ID NO: 36 - protein, 383 aa, synthetic construct
  REGION 1..383: note = Synthetic
  REGION 1..9: note = MISC_FEATURE - optionally absent
MHHHHHHGSSDEEEKKELEKRIEEAAQRAREAAERTGDPRVRELARELARLAERARELVERDPSSSDVNEALKLIVEAIEAAVRALEAAERAGDPELREDAREAVRLAVEAAEEVQRNPSSSTANLLLKAIVALAEALAAAANGDKEKFKKAAESALEIAKRVVEVASKEGDPEAVLEAAKVALRVAELAAKNGDKEVFKKAAESALEVAKRLVEVASKEGDPELVLEAAKVALRVAELAAKNGDKEVFQKAAASAVEVALRLTEVASKEGDSELETEAAKVITRVRELASKQGDAAVAILAETAEVKLEIEESKKRPQSESAKNLILIMQLLINQIRLLVLQIRMLDEQRQNQQREARVKSNEMERLAEVLRLSAREQRRAG

SEQ ID NO: 37 - protein, 383 aa, synthetic construct
  REGION 1..383: note = Synthetic
  REGION 1..9: note = MISC_FEATURE - optionally absent
MHHHHHHGSSDEEERKELEKRIREAAQRAREAAERTGDPRVRELARELARLAERARELVERDPSSSDVNEALKLIVEAIEAAVRALEAAERAGDPELREDAREAVRLAVEAAEEVQRNPSSSTANLLLKAIVALAEALAAAANGDKEKFKKAAESALEIAKRVVEVASKEGDPEAVLEAAKVALRVAELAAKNGDKEVFKKAAESALEVAKRLVEVASKEGDPELVLEAAKVALRVAELAAKNGDKEVFQKAAASAVEVALRLTEVASKEGDSELETEAAKVITRVRELASKQGDAAVAILAETAEVKLEIEESKKRPQSESAKNLILIMQLLINQIRLLVLQIRMLDEQRQRLEQQMRMEVRQLEIRSRCLQEESEVVEEVE

SEQ ID NO: 38 - protein, 267 aa, synthetic construct
  REGION 1..267: note = Synthetic
  REGION 1..9: note = MISC_FEATURE - optionally absent
MHHHHHHGSSEEEQERIRRILKEARKSGTEESLRQAIEDVAQLAKKSQDSEVLEEAIRVILRIAKESGSEEALRQAIRAVAEIAKEAQDSEVLEEAVRVIEEIAKESGSEEALRQAKRAIEEIAREARDLRVEALALLAMARLYLLMVKLEQEEKAKEFQELLKELSERSEELIRELEEKGAASEAELARMKQQHMTAYLEAQLTAWEIESKSKIALLELQQNQLNLELRLMKEILRRKEKALELRKLLLAAQALVQAAAQAERQTR

SEQ ID NO: 39 - protein, 137 aa, synthetic construct; identical to SEQ ID NO: 25
  REGION 1..137: note = Synthetic
  REGION 1..2: note = MISC_FEATURE - optionally absent

SEQ ID NO: 40 - protein, 137 aa, synthetic construct
  REGION 1..137: note = Synthetic
  REGION 1..2: note = MISC_FEATURE - optionally absent
MGSAEELLRRSREYLKKVKEEQERKAKEFQELLKELSERSEELIRELEEKGAASEAELARMKQQHMTAYLEAQLTAWEIESKSKIALLELQQNQLNLELRARALEAHLIALAARLKVEAAKAQAAADAIRKAAEEAR

SEQ ID NO: 41 - protein, 164 aa, synthetic construct
  REGION 1..164: note = Synthetic
  REGION 1..2: note = MISC_FEATURE - optionally absent
  REGION 157..164: note = MISC_FEATURE - optionally absent
MGSDEQDTLLDRMIREAAEAAKRALEAQARQQRTQSKDEAELAYLLGELAYKLGEYRIAIRAYRIALKRDPNNAEAWYNLGNAYYKQGDYDEAIEYYQKALELDPNNAEAWYNLGNAYYKQGDYDEAIEYYQKALELDPNNAEAKQNLGNAKQKQGGSHHHHHH

SEQ ID NO: 42 - protein, 164 aa, synthetic construct
  REGION 1..164: note = Synthetic
  REGION 1..2: note = MISC_FEATURE - optionally absent
  REGION 157..164: note = MISC_FEATURE - optionally absent
MGDHDAEEMFKRAAHASKRASKENADAAELLATAIAKDLAELAYLLGELAYKLGEYRIAIRAYRIALKRDPNNAEAWYNLGNAYYKQGDYDEAIEYYQKALELDPNNAEAWYNLGNAYYKQGDYDEAIEYYQKALELDPNNAEAKQNLGNAKQKQGGSHHHHHH

SEQ ID NO: 43 - protein, 164 aa, synthetic construct
  REGION 1..164: note = Synthetic
  REGION 1..2: note = MISC_FEATURE - optionally absent
  REGION 157..164: note = MISC_FEATURE - optionally absent
MGSSEAKELIEKALKNLLKIATKQAELQATIVKAQALDVAELAYLLGELAYKLGEYRIAIRAYRIALKRDPNNAEAWYNLGNAYYKQGDYDEAIEYYQKALELDPNNAEAWYNLGNAYYKQGDYDEAIEYYQKALELDPNNAEAKQNLGNAKQKQGGSHHHHHH

SEQ ID NO: 44 - protein, 164 aa, synthetic construct
  REGION 1..164: note = Synthetic
  REGION 1..2: note = MISC_FEATURE - optionally absent
  REGION 157..164: note = MISC_FEATURE - optionally absent
MGSEHNKDMITEALRVFEEAAEMAARAYKTLVTAQNQSVAELAYLLGELAYKLGEYRIAIRAYRIALKRDPNNAEAWYNLGNAYYKQGDYDEAIEYYQKALELDPNNAEAWYNLGNAYYKQGDYDEAIEYYQKALELDPNNAEAKQNLGNAKQKQGGSHHHHHH

SEQ ID NO: 45 - protein, 238 aa, synthetic construct
  REGION 1..238: note = Synthetic
  REGION 1..9: note = MISC_FEATURE - optionally absent
MHHHHHHGSDECEEKARRVAEKVERLKRSGTSEDEIAEEVAREISEVIRTLKESGSSYEVICECVARIVAEIVEALKRSGTSAVEIAKIVARVISEVIRTLKESGSSYEVICECVARIVAEIVEALKRSGTSAAIIALIVALVISEVIRTLKESGSSFEVILECVIRIVLEIIEALKRSGTSEQDVMLIVMAVLLVVLATLHREDQKVNNTALAIMMEALAEAAQLAAEAAKELKKSV

SEQ ID NO: 46 - protein, 159 aa, synthetic construct
  REGION 1..159: note = Synthetic
  REGION 152..159: note = MISC_FEATURE - optionally absent
SEEEARTIAKEAATAFAKLALLQAEAFATLVKAAARVAYILGAIAYAQGEYDIAITAYQVALDLDPNNAEAWYNLGNAYYKQGDYDEAIEYYQKALELDPNNAEAWYNLGNAYYKQGDYDEAIEYYQKALELDPNNAEAKQNLGNAKQKQGGSHHHHHH

SEQ ID NO: 47 - protein, 238 aa, synthetic construct
  REGION 1..238: note = Synthetic
  REGION 1..9: note = MISC_FEATURE - optionally absent
MHHHHHHGSDECEEKARRVAEKVERLKRSGTSEDEIAEEVAREISEVIRTLKESGSSYEVICECVARIVAEIVEALKRSGTSAVEIAKIVARVISEVIRTLKESGSSYEVICECVARIVAEIVEALKRSGTSAAIIALIVALVISEVIRTLKESGSSFEVILECVIRIVLEIIEALKRSGTSEQDVMLIVMAVLLVVLATLQTEILKAINHALAVMAQALAEAAQRAAEAAKKSATHI

SEQ ID NO: 48 - protein, 40 aa, synthetic construct
  REGION 1..40: note = Synthetic
  REGION 1..2: note = MISC_FEATURE - optionally absent
  REGION 33..40: note = MISC_FEATURE - optionally absent
MGTLVEILARAQIESSRVNIELAREALERAKRGSHHHHHH

SEQ ID NO: 49 - protein, 59 aa, synthetic construct
  REGION 1..59: note = Synthetic
  REGION 1..9: note = MISC_FEATURE - optionally absent
MHHHHHHGSSQEEESKRLVEEIAKRLKKIAEEIARAVEKLARAIIEALEVAWRSNKKIS

SEQ ID NO: 50 - protein, 157 aa, synthetic construct
  REGION 1..157: note = Synthetic
SEYEIRKALEELKAATAELKRATASLRAITEELKRLAKALAEKMYKAGNAMYRKGQYTIAIIAYTLALLADPNNAEAWYNLGNAAYKKGEYDEAIEAYQKALELDPNNAEAWYNLGNAYYKQGDYDEAIEYYQKALELDPNNAEAKQNLGNAKQKQG

SEQ ID NO: 51 - protein, 155 aa, synthetic construct
  REGION 1..155: note = Synthetic
ALVEHNRAIVEHNAIIVEHNRIIAAVLELIVRAIAHTAAELAYLLGELAYKLGEYRIAIRAYRIALKLDPNNAEAWYNLGNAYYKQGDYDEAIEYYQKALELDPNNAEAWYNLGNAYYKQGDYDEAIEYYQKALELDPNNAEAKQNLGNAKQKQG

(19) United States
(12) Patent Application Publication
(10) Pub. No.: US 2024/0010687 A1
(43) Pub. Date: Jan. 11, 2024

(54) De novo designed rotor (axle:ring) protein assemblies

(71) Applicant: University of Washington, Seattle, WA (US)

(72) Inventors: David Baker, Seattle, WA (US); Alexis Courbet, Seattle, WA (US); Jesse Hansen, Seattle, WA (US); Yang Hsia, Seattle, WA (US); Neville Bethel, Seattle, WA (US); Justin Kollman, Seattle, WA (US)

(21) Appl. No.: 17/931,214

(22) Filed: Sep. 12, 2022

Related U.S. Application Data
(60) Provisional application No. 63/246,045, filed on Sep. 20, 2021.

(51) Int. Cl. C07K 14/245

(52) U.S. Cl. CPC C07K 14/245

(57) ABSTRACT

Polypeptides that comprise axle or ring components of protein nanomachines, and kits and nanomachines including such polypeptides are disclosed herein.

### CROSS REFERENCE

This application claims priority to U.S. Provisional Patent Application Ser. No. 63/246,045 filed Sep. 20, 2021, incorporate by reference herein in its entirety.

### FEDERAL FUNDING STATEMENT

This invention was made with government support under Grant No. T32 GM008268, awarded by the National Institutes of Health and Grant No. CHE-1629214, awarded by the National Science Foundation. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Aug. 16, 2022 having the file name “21-1152-US.xml” and is 99 kb in size.

### BACKGROUND

The design of dynamic protein mechanical systems is of great interest given their rich functionality, but while recent advances in protein design permit the generation of somewhat sophisticated static nanostructures and assemblies, the complex folding and diversity of non-covalent interactions in dynamic protein mechanical systems has made their design very challenging.

### SUMMARY

In one aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-15 and 17-51, not including any functional domains added fused to 35 the polypeptides (whether N-terminal, C-terminal, or internal), and wherein the 1, 2, 3, 4, or 5 N-terminal and/or C-terminal amino acid residues may be present or absent and when absent are not considered in determining the percent identity.

In another embodiment, the disclosure provides kit or machine assemblies, comprising an axle and ring pair comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of one or more axle and ring pair are selected from the group consisting of the following pairs (A)-(J), not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity.
(A) SEQ ID NO:5 and SEQ ID NO:6;
(B) SEQ ID NO:7 and SEQ ID NO:8;
(C) SEQ ID NO:9 and SEQ ID NO:10;
(D) SEQ ID NO:11 and SEQ ID NO:12;
(E) SEQ ID NO:13 and SEQ ID NO:14;
(F) SEQ ID NO:15 and SEQ ID NO:17;
(G) SEQ ID NO:18 and SEQ ID NO:19;
(H) SEQ ID NO:20 and SEQ ID NO:21;
(I) SEQ ID NO:22 and SEQ ID NO:23; and/or
(J) SEQ ID NO:24 and SEQ ID NO:25.

The disclosure further provides nucleic acids encoding the polypeptides of the disclosure, expression vectors comprising the nucleic acids operatively linked to a suitable control sequence, host cells comprising the polypeptide, kits, machine assemblies, nucleic acids, and/or vectors of the disclosure; and methods for using the polypeptide, kits, machine assemblies, nucleic acids, vectors, and/or host cells of the disclosure.

### DESCRIPTION OF THE FIGURES

FIG. 1. Overview of rotary machine assembly and ring design approaches. (A) (Left) A blueprint of a simple rotary machine consisting of an assembly of an axle and a ring, mechanically constrained by the interface between the two; (Middle) Systematic generation of a structurally diverse library of machine components through computational design. The design of the interface between axle and ring mechanically couples the components by providing control on the rotational energy landscape and directing assembly; (Right) Example of hierarchical design and assembly of a protein rotary machine from axle and ring components, here a D3 axle and C3 ring, and interacting interface residues. Cyclic DHRs or wheels are fused to the end of the axle and ring components to increase mass, provide a modular handle and a rotation dependent structural signature. (B) Hierarchical design strategies for ring components (Top) A single chain C1 symmetric and internally C12 symmetric alpha-helical tandem repeat protein is split into three subunits, and each is fused to DHRs via helical fusion to generate a C3 ring with an internal diameter of 28 Å. The 6.5 Å cryoEM electron density shows agreement with the design model; (Middle) A single chain C1 symmetric and internally C24 symmetric alpha-helical tandem repeat protein is split into 4 subunits and each is fused to DHRs to generate a C4 ring with an internal diameter of 57 Å. The 5.9 Å cryoEM electron density shows agreement with the design model; (Bottom) Heterooligomeric helical bundles and DHRs are fused and assembled into a higher-ordered closed C3 structure through helical fusion (WORMS), after which another round of helical fusion protocol is used to fuse DHRs to each subunit, to generate a C3 ring with an internal diameter of 41 Å. The negative stain electron density shows agreement with the design model. Scale bar: 10 nm,

FIG. 2. Design of axle machine components. (A) Hierarchical design of a D3 symmetric homohexamer axle (1552_1na0C3_int2_11). Parametric design of interdigitated helices in D3 symmetry is achieved by sampling supercoil radius (R₁,R₂), helical phase (Δφ₁₋₁, Δφ₁₋₂), supercoil phase (Δφ₀₋₁, Δφ₀₋₂) of two helical fragments, and the z-offset (Z_{off}), and supercoil twist (ω₀). The interface is designed using the HBNet protocol to identify hydrogen-bond networks spanning the 6 helices mediating high-order specificity. The design is then fused to C3 homotrimers using Rosetta™ Remodel. The 4.2 Å cryoEM electron density is consistent with the design model (B) Hierarchical design of a D8 axle (D8A_1615). Starting from a parametrically designed C8 homohexamer, interdigitated helical extensions are sampled using Rosetta™ BluePrintBuilder and hydrogen bond networks identified using HBnet while sampling rotation and translation in D8 symmetry using Rosetta™ SymDofMover. The 5.9 Å cryoEM electron density shows close agreement with the design model; (C) Hierarchical design of a C3 homotrimer axle (A15.5). A parametrically designed C3 homotrimer is circularly permutated and an extra heptad repeat is added to increase the aspect ratio, after DHRs are fused to each subunit using Hfuse. The negative stain electron density is consistent with the design model (D) Additional axle designs (Top) Representative SEC, SAXS and negative stain EM profile corresponding to a D8 design (D8_6_49). The SAXS trace is similar to the computed trace from the model; (Bottom) Design models for D2_1119_7_tj81C2_V39_6, DC4G1_178, D5 _57C, and C8D8_6_49 overlaid with experimental 3D electron density. Scale bar: 10 nm.

FIG. 3. Design of symmetry mismatched D3-C3 and D3-C5 axle-ring assemblies. (A) Quasisymmetric axle and ring complex directed self-assembly strategy. Axles and rings are designed with complementary charged residues at their interfaces (electrostatic potential shown), buried histidine bond networks and disulfide bonds across the ring asymmetric unit interfaces to allow pH controlled assembly and oxidoreductive locking of the ring around the axle. Assembly monitored by negative stain EM (square panels) yields fully assembled rotors (cryoEM electron density on right). (B) Models of assemblies generated from a D3 axle (1552_1na0C3_int2_11) and C3 (R113) or C5 (C2arms9) rings, and cryoEM 2D average of axle alone before assembly. (C) Interface shape and symmetry results in different DOFs. In MD simulations, the D3-C3 system is largely constrained to rotation along the z axis, while the D3-C5 assembly allows rotation along x, y and z, and translation in z, x and y. (Left) N-C termini unit vectors of an ensemble of MD trajectories (Right) Vector magnitude corresponding to the computed mean square displacement of the ring relative to the axle along the 6 DOFs. (D) 3D CryoEM reconstruction of D3-C3 (Left) and D3-C5 (Right) rotors (axle as surface and ring as mesh, processed in D3 for D3-C3; processed in C1 and shown as surface and mesh at different thresholds for D3-C5; maps are shown as side view, end-on views and transverse slices) and experimental (top row) and theoretical 2D class averages with (middle row) and without (bottom row) explicitly sampling along DOFs. The D3-C3 rotor electron density at 10.2 Å resolution suggests that the ring sits midway across the D3 axle consistent with the designed mechanical DOF. The D3-C5 rotor cryoEM electron density at 11.4 Å captures the features of the designed structure also evident in the class average (Right). The 2D averages capture secondary structure corresponding to the C5 ring but could not be fully resolved, consistent with the ring populating multiple rotational states. Scale bar for cryoEM density: 10 nm.

FIG. 4. Computational sculpting of the rotational energy landscape by design of interface side-chain interactions. (A) Symmetry matched C3-C3 axle and ring complex (Left) Axle, ring, and rotor assembly models. The rotational energy landscape computed by scoring 10 independent Rosetta™ backbone and side-chains relax and minimization trajectories (solid line with error bars depicting the standard deviation) features three main energy minima corresponding to the C3 symmetry of the interface with 9 additional lesser energy minima. (Right) Single particle cryoEM analysis of the designed C3-C3 rotor. The electron density at 6.5 Å resolution shows the main features of the designed structure, evident in the experimental 2D class average (top row) compared to theoretical 2D class averages with (middle row) and without (bottom row) explicitly sampling the DOFs (B) Quasisimmetric D8-C4 axle and ring complex (Left) Axle, ring, and rotor assembly models. The rotational energy landscape computed as described in A features eight main energy minima corresponding to the C8 symmetry of the interface (Right) Single particle cryoEM analysis of the designed D8-C4 rotor. The electron density at ˜5.9 Å resolution shows the main features of the designed structure. (C) 3D variability analysis of the cryoEM data in relation with the rotational landscape of the D8-C4 rotary machine. The two distinctly resolved structures are separated by a 45° rotational step. Scale bar: 10 nm.

FIG. 5. Detail of the library of axle parts for the design of rotary machines with corresponding symmetry, design nomenclature, oligomeric mass, SEC chromatograms, SAXS traces, designed PDB model, and 3D electron density reconstruction from electron microscopy analysis. For each SEC trace, the theoretical elution volume corresponding to the correct oligomer state is given in milliliters next to the chromatogram. Experimental SAXS traces and the theoretical trace corresponding to the design are shown. nsEM: data obtained using negative stain electron microscopy; cryoEM: data obtained using single particle cryoelectron microscopy.

FIG. 6. Detail of the library of axle and ring parts for the design of rotary machines with corresponding symmetry, design nomenclature, oligomeric mass, SEC chromatograms, SAXS traces, designed PDB model, and 3D electron density reconstruction from electron microscopy analysis. For each SEC trace, the theoretical elution volume corresponding to the correct oligomer state is given in milliliters next to the chromatogram. Experimental SAXS traces and theoretical traces corresponding to the design are shown. nsEM: data obtained using negative stain electron microscopy; cryoEM: data obtained using single particle cryoEM.

FIG. 7. CryoEM data processing pipelines used to generate electron density and structures of the C3-C3 rotary machine. (A) Detail of the data processing pipeline (B) Representative cryoEM micrograph (C) FSC validation curve (D) Electron density map with corresponding estimated local resolution

FIG. 8. Detailed comparison of designs versus high resolution cryoEM structures. The designs were relaxed into experimental cryoEM electron densities using Rosetta™ FastRelax and SetupForDensityScoring. (A) D3 axle design (1552_1na0C3_int2_11); (Left) Superposition of the designed backbone and backbone relaxed into the experimental electron density, full structure and single chain alignment. The computed backbone atom RMSD from the designed and experimental structure is 1.930 Å. (Right) Detail of side chain density that becomes visible at this resolution (˜4 Å). (B) D8 axle design (D8A_1615). Superposition of the designed backbone and backbone relaxed into the experimental electron density, full structure and single chain alignment. The computed backbone atom RMSD from the designed and experimental structure is 2.879 Å. (C) C3 ring design (R82). Superposition of the designed backbone and backbone relaxed into the experimental electron density, full structure and single chain alignment. The computed backbone atom RMSD from the designed and experimental structure is 3.451 Å.

FIG. 9. CryoEM data processing pipelines used to generate electron density and structures of the D8-C4 rotary machine. Interestingly, this design self-assembled into higher-order fiber-like structures upon freezing, which highlights the unintended effects of cryogenic conditions on protein assemblies. We could however verify that this fiber assembly did not happen at room temperature in solution, as can be seen from SAXS, SEC in FIG. 7, as well as negative stain EM. (A) Detail of the data processing pipeline (B) Representative cryoEM micrograph. Top: Negative stain; bottom: cryoEM. Freezing conditions seemed to induce fiber formation via end-to-end contact of the D8 axle. (C) FSC validation curve (D) Electron density map with corresponding estimated local resolution.

FIG. 10. CryoEM data processing pipelines used to generate electron density and a structure of the D3 axle. This data was collected on a version of the D3-C5 rotary machine, for which the C5 ring did not have arms extension, thus precluding obtention of clear ring density. We used this dataset to thus focus on obtaining a clear picture of the axle, as detailed here. (A) Detail of the data processing pipeline (B) Representative cryoEM micrograph (C) FSC validation curve (D) Electron density map with corresponding estimated local resolution.

FIG. 11. Chemical synthesis of a 36 residues helical peptide self-assembling into a D3 homohexamer. (A) HPLC chromatogram post synthesis, showing two elution peaks. (B) Deconvoluted native mass spectra corresponding to the HPLC peaks. (C) Size exclusion chromatography (top, 215nm absorbance) coupled with multiple angle laser light scattering analysis (bottom) of the collected fractions post synthesis and purification. Integration of peak two gives a molecular weight of 25 kDa +/−7, corresponding to the size of the homohexamer assembly.

FIG. 12. Detail of the library of fully assembled rotary machines with corresponding symmetry, design nomenclature, oligomeric mass, SEC chromatograms, SAXS traces, designed PDB model, and 3D electron density reconstruction from electron microscopy analysis. For each SEC trace, the theoretical elution volume corresponding to the correct oligomer state is given in milliliters in black next to the chromatogram. Experimental SAXS traces and the theoretical traces corresponding to the design are shown. nsEM: data obtained using negative stain electron microscopy; cryoEM: data obtained using single particle cryoelectron microscopy.

FIG. 13. Biolayer interferometry assays measuring in vitro assembly of ring and axle parts into a full rotary system. For each 6 designs, the equilibrium binding curves from biolayer interferometry binding assays is shown on the left and the corresponding Biolayer interferometry kinetic binding traces shown on the right. Biotinylated axles were immobilized on the tip and the binding in a solution of fre rings was measured. For both D3-C5 and D3-C3, a fresh ring solution was prepared by buffer exchange from citrate buffer to TBS with reducing agent, and immediately used for binding assays.

FIG. 14. Example of designed energy landscapes. The shape, periodicity and energetics differ drastically depending on the residue identities and contact types for the same protein scaffold. (Top) Two C3-C3 rotors design trajectories; (Bottom) Two C5-C3 rotors design trajectories: C5C3_3250, C5C3_2412; (Left) PDB models \; (Right) Energy landscapes shown as polar maps, depicting Rosetta™ Energy Units (REU) vs rotation angle generated by sampling along the rotational degree of freedom while using Rosetta™ relax, minimization of side chains and scoring for each rotation bins. The mean energy landscape obtained from 10 independent trajectories is shown in red with error bars depicting the standard deviation. The designed interface between axle and ring at angle=0 is shown beside as cross-sections, showing residue identities and contacts and hydrogen-bond networks with the helical backbone.

FIG. 15. In vivo assembly of two component rotary machines from bicistronically expressed axle and ring parts (A) Plasmid architecture for the bicistronic expression system based pET29b+. (B) SDS-PAGE after Ni-NTA purification while bicistronically expressing axle and ring in the same cell. A single band indicates that the ring did not pull down the axle (lanes marked with circle), while 2 bands indicate assembly of axle and ring (marked as an triangle) (C) Convoluted and deconvoluted native mass spectrums of the isolated C3-C3 rotary machine. (D) SAXS traces of the purified protein. The experimental traces and the theoretical traces corresponding to the design are shown.

FIG. 16. CryoEM data processing pipelines used to generate electron density and structures of the D3-C3 rotary machine. (A) Detail of the data processing pipeline. The Cl reconstruction in stain yielded C3 features, which allowed us to further process the design with C3 symmetry imposed here. The C3 reconstruction also showed ring density that was polar, consistent with the rotor design. The ring density looks very similar when processed in D3, while yielding a better model for the axle (which has D3 symmetry by design). Therefore we used a whole model processed in D3 mode to present in FIG. 3C, which is closest to the actual symmetry and structure of the model (B) Representative cryoEM micrograph (C) FSC validation curve for C3 reconstruction. (D) Electron density map with corresponding estimated local resolution.

FIG. 17. CryoEM data processing pipelines used to generate electron density and structures of the D3-C5 rotary machine. (A) Detail of the data processing pipeline (B) Representative cryoEM micrograph (C) FSC validation curve of C1 reconstruction (D) Electron density map with corresponding estimated local resolution.

FIG. 18. Detail of DOF sampling for the generation of the theoretical cryoEM 2D class averages projections compared to experimental data and models. (A) C3-C3 rotor (top) Experimental 2D averages (middle) Projections obtained when taking into account the rotational DOF and simulating 10 trajectories with corresponding PDB model shown on the left (bottom) Projections obtained when not taking into account the rotational DOF with corresponding PDB model shown on the left (B) D3-C3 (top) Experimental 2D averages (middle) Projections obtained when taking into account the rotational DOF and simulating 10 trajectories with corresponding PDB model shown on the left (bottom) Projections obtained when not taking into account the rotational DOF and simulating 10 trajectories with corresponding PDB model shown on the left (C) D3-C5 rotor (top) Experimental 2D averages (middle) Projections obtained when taking into account the rotational and translational DOF and simulating 10 trajectories with corresponding PDB model shown on the left (bottom) Projections obtained when not taking into account the rotational and translational DOFs and simulating 10 trajectories with corresponding PDB model shown on the left.

FIG. 19. Molecular dynamics simulations performed on D3-C3 and D3-C5 rotary machine assemblies to investigate the DOF of motion (A) The interface shape, size and symmetry of these two design results in different DOFs: the D3-C3 was found to rotate along the z axis, while the D3-C5 ring showed rotation along x, y and z, as well as translation in z and y. The top panel shows z translation of rings relative to rotation, the middle panel shows the x and y rotation or the ring, or tilt, relative to rotation and the bottom panel shows the x, y translation of the ring relative to the rotation around the axle. (B) Top: D3-C3; Bottom: D3-C5; Left: PDB models; Right: density maps of the backbone atoms showing averaged motion of ring and axle relative to each other.

FIG. 20. Rotary machine modular extension by systematic fusion with reversible heterodimers. (Left) SEC elution profiles corresponding the C3-C3 rotor assembly with and without heterodimer arms extension, or ring only with or without heterodimer extension. (Right) Top and side views of negative stain 3D reconstruction corresponding to the ring only with or without the heterodimer arms.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-15 and 17-51, not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity.

The polypeptides disclosed herein are de novo proteins designed as single components (axles/rings) and full rotary machine assemblies, and this can be used, for example, in protein nanomachines that be genetically encoded for multicomponent self-assembly within cells or in vitro, facilitating fabrication or in vivo transfer and use in a vast range of nanodevices for medicine, material sciences or industrial bioprocesses.

The sequences provided below are annotated as follows:
Single-underlined residues: Interface residues needed for two component interaction mediating rotary machine assembly
Bolded residues: Structural residues supporting axle assembly
Double underlined residues: Modular designed helical repeat domains (DHR domains), can be exchanged for any other DHR sequence as deemed suitable (including but not limited to DHR82, DHR53, DHR20, and DHR15).

>DHR82
(SEQ ID NO: 1)
AYALELALGALRLEDRARELIKEAEKKGDPEKLREALEALEEAVRLVEEAIKLRPDMDLAVEIAVRLARMLKRV
AELLQELAKKTGDPELLKLALRALEVAVRAVELAIKSNPDNDEAVETAVRLARELKKVAEELQERAKKTGDPEL
LKLALRALEVAVRAVELAIKSNPDNEEAVETAKRLAEELRKVAELLEERAKETGDPELQELAKRAKEVADRARE
LAKKS
>DHR53
(SEQ ID NO: 2)
NDEKEKLKELLKRAEELAKSPDPEDLKEAVRLAEEVVRERPGSNLAKKALEIILRAAEELAKLPDPEALKEAVK
AAEKVVREQPGSNLAKKALEIILRAAAALANLPDPESRKEADKAADKVRRE
>DHR20
(SEQ ID NO: 3)
ELAKRADDKDVREIVRDALELASRSTNDEVIRLALEAAVLAARSTDSDVLEIVKDALELAKQSTNEEVIKLALK
AAVLAAKSTDEEVLEEVKEALRRAKESTDEEEIKEELRKAVEEAE
>DHR15
(SEQ ID NO: 4)
DERQKQREEVRKLAEELASKATDEELIKEIKKCAQLAEELASRSTNDELIKQILEVAKLAFELASKATDEELIK
RILKCCQLAFELASRSTNDELIKQILEVAKLAFELASKATDEELIKLILACCVLAFELASRITNDEEIKQILEE
AKEAFERASKATDEEEIRKILAKCIA

Residues within squiggly brackets: {Residues needed for binding to small molecule (fuel/inhibitor) to produce torque/lock the rotor}
Residues in brackets: [Loop regions subject to modifications]
Residues in parentheses: Optional residues that may be present or absent(Facultative affinity purification tags and linkers)
Axle and Ring denote the two different asymmetric units in a two component rotary machine assembly (see below).

Full Rotary machine assemblies:

>A113_c2arms9_Ring (D3-C5)
(SEQ ID NO: 5)
(MG)DRSEHAKKLKTFLENLRRHLDRLDKHIKQLRDILS[ENPEDER]VKDVIDLSERSVRIVKTVIKIFEDSVREL
EKAILWLAEELAKSPDPEDLKRAVELARAVIEANPGSNLSRKAMEIIERAARELSKLPDPEAQRTAIEAASQLATMA
AATGNTDQVRRAAELMVEIARLAGTEEAQDLALDALLDVLETALQIATKIIDDANKLLEKLR[RSERKDP]KVVETY
VELLKRHEEAVRLLLEVARVHEELVRFTIIEEKVRSPDCEDIRDAVREAEELLRENPSEMAEELLRRAIEAAVRCPD
CEAIREAVRAAEELLRENPSTEAEELLRRAIEAAVRCPDCEAIREAVRAAEELLRENPSEEAKELLRRAIESAKKCP
DPEAQREAKRAEEELRKE(GSHHHHHH)
>A113_c2arms9_Axle (D3-C5)
(SEQ ID NO: 6)
DEEDESYELVEHIAEELEEIAEEIAEAVENLAQAIIEALYVAWESNQQINEQVQEVEQSMAELAYLLGELAYKLGEY
RIAIRAYRIALKHDPNNAEAWYNLGNAYYKQGDYDEAIEYYQKALELDPNNAEAWYNLGNAYYKQGDYDEAIEYYQK
ALELDPNNAEAKQNLGNAKQKQG(GSHHHHHH)
>A15.5R82_Axle (C3-C3)
(SEQ ID NO: 7)
(MG)DIEEAKEESRKIADHGHDGHKAVADLQRLNIE{LAHKLLDEVEQLQNLNDELARE}LLDLVDRLAELLIDLVR
KTSELTDEDTIRREILKVDVRMLAISLA[ASAKDEE]LRKEIKKCLQLAEELASRSTNKELQKQAMEVAKLALELA
[RKATDE]ELIKEILKCCQLAFELASRSTNDELIKQILEVAKLAFELA[SKATDEE]LIKEILKCCQLAFELASRSTN
DEEIKQILETAKEAFERAS[KATDEE]EIKEILKKCQEKFEKKS(GSHHHHHH)
>A15.5R82_Ring (C3-C3)
(SEQ ID NO: 8)
(MG){VEELLLLARAAHH}[SGTTVEE]AYKLAK[KLGISV]{KELLLLARAAHN}[SGTTVEE]AYKLA[LKLGIS]
{VEELLLLAKAAHY}[SGTTVE]EAY[KLALELGISV]{RELLLLAKAAHF}[AGRTVRE]AYALELALGALRLED
RARELIKEAEKKGDPEKLREALEALEEAVRLVEEAIKLRPDMDLAVEIAVRLARMLKRVAELLQELAKKTGDPELLK
LALRALEVAVRAVELAIKSNPDNDEAVETAVRLARELKKVAEELQERAKKTGDPELLKLALRALEVAVRAVELAIKS
NPDNEEAVETAKRLAEELRKVAELLEERAKETGDPELQELAKRAKEVADRARELAKKS(GSHHHHHH)
>C3D3_AR113_Axle (D3-C3)
(SEQ ID NO: 9)
(MG)DEEDESYELVEHIAEELEEIAEEIAEAVENLAQAIIEALYVAWESNQQINEQVQEVEQSMAELAYLLGELAYK
LGEYRIAIRAYRIALKHDPNNAEAWYNLGNAYYKQGDYDEAIEYYQKALELDPNNAEAWYNLGNAYYKQGDYDEAIE
YYQKALELDPNNAEAKQNLGNAKQKQG(GSHHHHHH)
>C3D3_AR113_Ring (D3-C3)
(SEQ ID NO: 10)
(MG)QRSGHARQLKRHLHRLRRHLERLDKHAKHLRQILSERPQDERVKDSIDLLEESVRIVKISIKIFEASVRALLW
AINKEAEELAKSPDPHDLHRAVRLARAVVQADPGSNLSKKALEIILRAAAELAKLPDPNALAAAARAASQVQREQPG
SNLAKAAQEIMRQASRAAEEAARRAKETLEKAEKRGDPKTALQAVRTVVKVAAALNQIATMAGSEEAQERAARVAAE
AAELALRVFELAEKQGDPHVARRARKLIQTVLQILLRILTQILETATKIIEEANKLLRKHRRSSRKDPKLVETHVEL
VKRHERLVRQHLKIALMHALAVLELAFPDAEAAKLASKAAKEAEELCKQSTDERLCDLLAELAALLIELAARYPDSE
AAKLALKAALEAIELCKQSTDEELCEELVKLAQKLIELAKRYPDSEEAKRALKEAKELIEQCKESTDEDECRELVKR
AEELIREAKE(GSHHHHHH)
>C5C3_2412_Axle (C5-C3)
(SEQ ID NO: 11)
(MHHHHHHGS)SDEEEKKELEKRIEEAAQRAREAAERTGDPRVRELARELARLAERARELVERDPSSSDVNEALKLI
VEAIEAAVRALEAAERAGDPELREDAREAVRLAVEAAEEVQRNPSSSTANLLLKAIVALAEALAAAANGDKEKFKKA
AESALEIAKRVVEVASKEGDPEAVLEAAKVALRVAELAAKNGDKEVEKKAAESALEVAKRLVEVASKEGDPELVLEA
AKVALRVAELAAKNGDKEVFQKAAASAVEVALRLTEVASKEGDSELETEAAKVITRVRELASKQGDAAVAILAETAE
VKLEIEESKKRPQSESAKNLILIMQLLINQIRLLVLQIRMLDEQRQNQQREA{RVKSNEMERLAEVLRLSARARRGA
MSGSEEDQERLRKEMEEERKHMEEVEK}ELRKVEEKMKSHEDTSL{RLLVLIARLLINQIRLLILQIRSLSNLERNQ
AREAMVESNEMEREAETLRLSAR}EQRRAG
>C5C3_2412_Ring (C5-C3)
(SEQ ID NO: 12)
(MG){DRSGHAKKLKTHLENLRRHLDRLDKHAKQLRDILSEH}PHDERVKDSIDLLEESVRIVKISIKIFEASVRAL
LWAINKEAEELAKSPDP{EDLKRAVELAEAVVR}ADPGSNLSKKALEIILRAAAELAKLPDP{DALAAAARAASKVQ
Q}EMPGSNLAKAAQEIMRQASRAAEEAARRAKETLEKAEKDGDP{ETALKAVETVVKVARALNQIATA}AGSEEAQE
RAARVAAEAAELALRVFELAEKQGDP{EVARRARELIEKVLDLLLSLLTQILQTATKVIDDSNKLLEKLRR}SHHHD
PKLVETHVELVKRHERLVRQHLKIALMHALAVLELAFPDAEAAKLASKAAKEAEELCKQSTDERLCDLLAELAALLI
ELAARYPDSEAAKLALKAALEAIELCKQSTDEELCEELVKLAQKLIELAKRYPDSEEAKRALKEAKELIEQCKESTD
EDECRELVKRAEELIREAKE(GSHHHHHH)
>C5C3_3250_Axle (C5-C3)
(SEQ ID NO: 13)
(MHHHHHHGS)SDEEERKELEKRIREAAQRAREAAERTGDPRVRELARELARLAERARELVERDPSSSDVNEALKLI
VEAIEAAVRALEAAERAGDPELREDAREAVRLAVEAAEEVQRNPSSSTANLLLKAIVALAEALAAAANGDKEKFKKA
AESALEIAKRVVEVASKEGDPEAVLEAAKVALRVAELAAKNGDKEVFKKAAESALEVAKRLVEVASKEGDPELVLEA
AKVALRVAELAAKNGDKEVFQKAAASAVEVALRLTEVASKEGDSELETEAAKVITRVRELASKQGDAAVAILAETAE
VKLEIEESKKRPQSESAKNLILIMQLLINQIRLLVLQIRMLDEQRQRLEQQM{RMEVRQLEIRSECLRKESAVVSMV
NSVGTHDQMKLKEQMEEEERHTEKVEK}EIRKVEEKMKSHEDTSLRLLVLIA{RLLINQIRLLILQIRSLSNLELRL
QQQMRMEVEQLRIRSQCLQEE}SEVVEEVE
>C5C3_3250_Ring (C5-C3)
(SEQ ID NO: 14)
(MG){NRSLHANKLKTHLENLREHLKRLDEHAKQLRDILSEH}PHDERVKDSIDLLEESVRIVKISIKIFEASVRAL
LWAINKEAEELAKSPDP{ADLERAVRLAAAVVR}ADPGSNLSKKALEIILRAAAELAKLPDP{KALAAAAEAASRVQ
R}EQPGSNLAKAAQEIMRQASRAAEEAARRAKETLEKAEKDGDP{RTALQAVMTVVEVAKALNIIATM}AGSEEAQE
RAARVAAEAAELALRVFELAEKQGDP{EVAHNARKLIEIVLHILLQILTQILETATKIIREANELLEKHRR}SHHHD
PKLVETHVELVKRHERLVRQHLKIALMHALAVLELAFPDAEAAKLASKAAKEAEELCKQSTDERLCDLLAELAALLI
ELAARYPDSEAAKLALKAALEAIELCKQSTDEELCEELVKLAQKLIELAKRYPDSEEAKRALKEAKELIEQCKESTD
EDECRELVKRAEELIREAKE(GSHHHHHH)
>C8D8_6 _49_119RC4_20_Axle (C8-C4)
(SEQ ID NO: 15)
(MHHHHHHGS)SEEEQERIRRILKEARKSGTEESLRQAIEDVAQLAKKSQDSEVLEEAIRVILRIAKESGSEEALRQ
AIRAVAEIAKEAQDSEVLEEAVRVIEEIAKESGSEEALRQAKRAIEEIAREARDLRVEALALLAMARLYLLMVKLEQ
EEKAKEFQELLKELSERSEELIRELEEKGAASEAELARMKQQHMTAYLEAQLTAWEIESKSKIALLELQQNQLNLEL
RLMKEILRRKEKALELRKLLLAAQALVQAAAQAERQTREDDSLREAEELLRRSREYLKKVKEEQERKAKEFQELLKE
LSERSEELIRELEEKGAASEAELARMKQQHMTAYLEAQLTAWEIESKSKIALLELQQNQLNLELRLHEAQKRRKEKA
LELRKLLLAAQALVQAAAQAERQTR
>C8D8_6_49_119RC4_20_Ring (C8-C4)
(SEQ ID NO: 17)
(MG)CDAIQAAAALGE[AGISS]NEILELLAAAAE[LGLDP]DAIQAAAQLGE[AGISS]EEILELLRAAHE[LGLD
P]DAIAAAADLGQ[AGISS]EEILELLRAAHELGLDPDAIQAAAALGE[AGISS]EEILELLRAAHE[LGLDP]DAI
QAAAQLGE[AGISS]EEILELLRAAHE[LGLDP]DCIAAAADLGQ[AGISS]SEITALLLAAAAIELAKRADDKDVR
EIVRDALELASRSTNDEVIRLALEAAVLAARSTDSDVLEIVKDALELAKQSTNEEVIKLALKAAVLAAKSTDEEVLE
EVKEALRRAKESTDEEEIKEELRKAVEEAE(GSHHHHHH)
>62.7_20_Axle
(SEQ ID NO: 18)
(MG)SIEEAEEESRKIAD[KGSDGH]KAVADLQRLNIKLAEDLLRHVEELQELNIDLARQLLRLVEELQKLNIDLVR
KTSELTDEKTIREEIRKVKEKSKEIV
>62.7_20_Ring
(SEQ ID NO: 19)
(MG)VEELLLLARAAHY[SGTTVEE]AYKLAL[KLGIS]VEELLLLARAAHQ[SGTTVEE]AYKLAL[KLGISV]KE
LLLLAQAARN[SGTTVEE]AYKLAL[KLGIS]VEELLLLAKAADF[SGTTVEE]AYKLAL[KLGIS]VEELLLLARA
AHY[SGTTVEE]AYKLAL[KLGIS]VEELLLLARAAHQ[SGTTVEE]AYKLAL[KLGIS]VKELLLLAQAARN[SGT
TVEE]AYKLAL[KLGIS]VEELLLLAKAADF[SGTTVEE]AYKLAL[KLGIS]VEELLLLARAAHY[SGTTVEE]AY
KLAL[KLGIS]VEELLLLARAAHQ[SGTTVEE]AYKLAL[KLGIS]VKELLLLAQAARN[SGTTVEE]AYKLAL[KL
GIS]VEELLLLAKAADF[SGTTVEE]AYKLAL[KLGIS](GSHHHHHH)
>54.7_112_Axle
(SEQ ID NO: 20)
(MG)DIEEAKEESRKIAD[HGHDGH]KAVADLQRLNIELA{HKLLDEVEQLQNLNIELARDL}LRLVEELQRLNIDL
VRKTSELTDEKTIREEIRKVKEESKRIVEEAEEEI
>54.7_112_Ring
(SEQ ID NO: 21)
(MG){VEELLLLARAAHH}[SGTTVEE]AYKLAL[KLGISV]{KELLLLARAAHN}[SGTTVEE]AYKLA[LKLGIS]
{VEELLLLAKAAHY}[SGTTVE]EAYKLAL[KLGISV]{RELLLLAKAAHF}[SGTTVE]EAYKLAL[KLGIS]{V
EELLLLARAAHH}[SGTTVEE]AYKLAL[KLGISV]{KELLLLARAAHN}[SGTTVE]EAYKLAL[KLGIS]{VEEL
LLLAKAAHY}[SGTTVEE]AYKLAL[KLGIS]{VRELLLLAKAAHF}[SGTTVEE]AYKLAL[KLGIS]{VEELLLL
ARAAHH}[SGTTVEE]AYKLAL[KLGISV]{KELLLLARAAHN}[SGTTVEE]AYKLAL[KLGIS]{VEELLLLAKA
AHY}[SGTTVEE]AYKLAL[KLGIS]{VRELLLLAKAAHF}[SGTTVEE]AYKLA[LKLGIS](GSHHHHHH)
>31.4_1_Axle
(SEQ ID NO: 22)
(HHHHHHMG)TEDLKYSLERLREILERLEENPSEKQIVEAIRAIVENNAQIVEAIRAIVDILRLIVSNNAAIVAILA
LIVDNNRAIVEILALIVENNRAIIEALEAIGGGTKILEEMKKQLKDLKRALET
>31.4_1_Ring
(SEQ ID NO: 23)
(MG)VEELLMLAIAAAASGTTVEEAYKLALKLGISVTELLALAAAAAASGTTVEEAYKLALKLGISVEELLMLAQAA
AFSGTTVEEAYKLALKLGIS(GSHHHHHH)
>119RC4_20_Ring (D8-C4)
(SEQ ID NO: 24)
(MG)CDAIQAAAALGE[AGISS]NEILELLAAAAE[LGLDP]DAIQAAAQLGE[AGISS]EEILELLRAAHE[LGLD
P]DAIAAAADLGQ[AGISS]EEILELLRAAHELGLDPDAIQAAAALGE[AGISS]EEILELLRAAHE[LGLDP]DAI
QAAAQLGE[AGISS]EEILELLRAAHE[LGLDP]DCIAAAADLGQ[AGISS]SEITALLLAAAAIELAKRADDKDVR
EIVRDALELASRSTNDEVIRLALEAAVLAARSTDSDVLEIVKDALELAKQSTNEEVIKLALKAAVLAAKSTDEEVLE
EVKEALRRAKESTDEEEIKEELRKAVEEAE(GSHHHHHH)
>119RC4_20_Axle (D8-C4)
(SEQ ID NO: 25)
(MG)SAEELLRRSREYLKKVKEEQERKAKEFQELLKELSERSEELIRELE[EKGAASEAE]LARMKQQHMTAYLEAQ
LTAWEIESKSKIALLELQQNQLNLELRLHEAQKRRKEKALELRKLLLAAEALVEAARQAERETR
Single components:
(SEQ ID NO: 26)
>1552_1na0C3_int2_11_Homohexameric D3 symmetric axle
(MG)SEEEESKRLVEEIAKRLKKIAEEIARAVEKLARAIIEALEVAWRSNKKINEQVQRVEQSMAELAYLLGELAYK
LGEYRIAIRAYRIALKHDPNNAEAWYNLGNAYYKQGDYDEAIEYYQKALELDPNNAEAWYNLGNAYYKQGDYDEAIE
YYQKALELDPNNAEAKQNLGNAKQKQG(GSHHHHHH)
>1na0C33_DSS310_20 Homohexameric D3 symmetric axle
(SEQ ID NO: 27)
(MG)TLVEILARAQIESSRVNIELAREALERAKHLHREAKGLAEKMYKAGNAMYRKGQYTIAIIAYTLALLSDPNNA
EAWYNLGNAAYKKGEYDEAIEAYQKALELDPNNAEAWYNLGNAYYKQGDYDEAIEYYQKALELDPNNAEAKQNLGNA
KQKQG(GSHHHHHH)
>A15.5 Homotrimeric C3 symmetric axle
(SEQ ID NO: 28)
(MG)DIEEAKEESRKIADHGHDGHKAVADLQRLNIELAHKLLDEVEQLQNLNDELARELLDLVDRLAELLIDLVRKT
SELTDEDTIRREILKVDVRMLAISLAASAKDEELRKEIKKCLQLAEELASRSTNKELQKQAMEVAKLALELARKATD
EELIKEILKCCQLAFELASRSTNDELIKQILEVAKLAFELASKATDEELIKEILKCCQLAFELASRSTNDEEIKQIL
ETAKEAFERASKATDEEEIKEILKKCQEKFEKKS(GSHHHHHH)
>C2arms9 Homopentameric C5 symmetric ring
(SEQ ID NO: 29)
(MG)DRSEHAKKLKTFLENLRRHLDRLDKHIKQLRDILSENPEDERVKDVIDLSERSVRIVKTVIKIFEDSVRELEK
AILWLAEELAKSPDPEDLKRAVELARAVIEANPGSNLSRKAMEIIERAARELSKLPDPEAQRTAIEAASQLATMAAA
TGNTDQVRRAAELMVEIARLAGTEEAQDLALDALLDVLETALQIATKIIDDANKLLEKLRRSERKDPKVVETYVELL
KRHEEAVRLLLEVARVHEELVRFTIIEEKVRSPDCEDIRDAVREAEELLRENPSEMAEELLRRAIEAAVRCPDCEAI
REAVRAAEELLRENPSTEAEELLRRAIEAAVRCPDCEAIREAVRAAEELLRENPSEEAKELLRRAIESAKKCPDPEA
QREAKRAEEELRKE(GSHHHHHH)
>C5_41 Homopentameric C5 symmetric axle
(SEQ ID NO: 30)
(MHHHHHHGS)SDEEEKKELEKRIEEAAQRAREAAERTGDPRVRELARELARLAERARELVERDPSSSDVNEALKLI
VEAIEAAVRALEAAERAGDPELREDAREAVRLAVEAAEEVQRNPSSSTANLLLKAIVALAEALAAAANGDKEKFKKA
AESALEIAKRVVEVASKEGDPEAVLEAAKVALRVAELAAKNGDKEVFKKAAESALEVAKRLVEVASKEGDPELVLEA
AKVALRVAELAAKNGDKEVFQKAAASAVEVALRLTEVASKEGDSELETEAAKVITRVRELASKQGDAAVAILAETAE
VKLEIEESKKRPQSESAKNLILIMQLLINQIRLLVLQIRMLDEQRQNQQREARVKSNEMERLAESLRLSARDRRGAM
SGSEEDQERIRKRMEEEEKDAEKVEKELRKVEEKMKSHEDTSLRLLVLIARLLINQIRLLILQIRSLSNLERNQARE
AMVHSNEMERRAEVLRLSAREQRRAG
>C6D3_50_Homohexameric D3 symmetric axle
(SEQ ID NO: 31)
(MHHHHHHGS)TEDEIRKLRKLLEEAEKKLKKLEDKTRRSEEISKTDDDPKAQSLQLIAESLMLIAESLLIIAISLL
LSSAGSTGAEDEIRKLRKLLEEAEKKLKKLEDKTRRSEEISKTDDDPKAQSLQLIAESLMLIAESLLIIAISLLLLA
EQAAREARIKERVKHAAEKMVRAAEAQAEFARLRAQ
>C8D8_6_49_C8 Homooctameric C8 symmetric axle
(SEQ ID NO: 32)
(MHHHHHHGS)SEEEQERIRRILKEARKSGTEESLRQAIEDVAQLAKKSQDSEVLEEAIRVILRIAKESGSEEALRQ
AIRAVAEIAKEAQDSEVLEEAVRVIEEIAKESGSEEALRQAKRAIEEIAREARDLRVEALALLAMARLYLLMVKLEQ
EEKAKEFQELLKELSERSEELIRELEEKGAASEAELARMKQQHMTAYLEAQLTAWEIESKSKIALLELQQNQLNLEL
RLMKEILRRKEKALELRKLLLAAQALVQAAAQAERQTREDDSLREAEELLRRSREYLKKVKEEQERKAKEFQELLKE
LSERSEELIRELEEKGAASEAELARMKQQHMTAYLEAQLTAWEIESKSKIALLELQQNQLNLELRLHEAQKRRKEKA
LELRKLLLAAQALVQAAAQAERQTR
>D2_1119_7 Homotetrameric D2 symmetric axle
(SEQ ID NO: 33)
(MG)DKAERSLDKQRRVAEELQKIIEKLQRAVKELQDVLETLKKVSTEQDRTTK(GSHHHHHH)
>D2_1119_7_tj81C2_V39_6 Homotetrameric D2 symmetric axle
(SEQ ID NO: 34)
(MHHHHHHGS)EREELSELAERILQKARKLSEEARERGDLKELALALILEALAVLLLAIAALLRGNSEEAERASEKA
QRVLEEARKVSEEAREQGDDEVLALALIAIALAVLALALVACSRGNSEEAERASEKAQRVLEEARKVSEEAREQGDD
EVLALALIAIALAVLALAIVASCRGNKEEAERAAEDAIKVAMEALEVLLSAVEQGDLKVALAAVIAILLAIAALLMV
IIKRRQDEKMERSLDKQRRVAEELQKIIEKLQRAVKELQDVLETLKKVSTEQDRTTK
>D4_1550_700 Homooctameric D4 symmetric axle
(SEQ ID NO: 35)
(MG)TEDELKERQDRLIEKFIKAMAKAASAHAELMRINSELVSR(GSHHHHHH)
>D5_41 Homodecameric D5 symmetric axle
(SEQ ID NO: 36)
(MHHHHHHGS)SDEEEKKELEKRIEEAAQRAREAAERTGDPRVRELARELARLAERARELVERDPSSSDVNEALKLI
VEAIEAAVRALEAAERAGDPELREDAREAVRLAVEAAEEVQRNPSSSTANLLLKAIVALAEALAAAANGDKEKFKKA
AESALEIAKRVVEVASKEGDPEAVLEAAKVALRVAELAAKNGDKEVFKKAAESALEVAKRLVEVASKEGDPELVLEA
AKVALRVAELAAKNGDKEVFQKAAASAVEVALRLTEVASKEGDSELETEAAKVITRVRELASKQGDAAVAILAETAE
VKLEIEESKKRPQSESAKNLILIMQLLINQIRLLVLQIRMLDEQRQNQQREARVKSNEMERLAEVLRLSAREQRRAG
>D5_57C Homodecameric D5 symmetric axle
(SEQ ID NO: 37)
(MHHHHHHGS)SDEEERKELEKRIREAAQRAREAAERTGDPRVRELARELARLAERARELVERDPSSSDVNEALKLI
VEAIEAAVRALEAAERAGDPELREDAREAVRLAVEAAEEVQRNPSSSTANLLLKAIVALAEALAAAANGDKEKFKKA
AESALEIAKRVVEVASKEGDPEAVLEAAKVALRVAELAAKNGDKEVEKKAAESALEVAKRLVEVASKEGDPELVLEA
AKVALRVAELAAKNGDKEVFQKAAASAVEVALRLTEVASKEGDSELETEAAKVITRVRELASKQGDAAVAILAETAE
VKLEIEESKKRPQSESAKNLILIMQLLINQIRLLVLQIRMLDEQRQRLEQQMRMEVRQLEIRSRCLQEESEVVEEVE
>D8_6_49 Homo16meric D8 symmetric axle
(SEQ ID NO: 38)
(MHHHHHHGS)SEEEQERIRRILKEARKSGTEESLRQAIEDVAQLAKKSQDSEVLEEAIRVILRIAKESGSEEALRQ
AIRAVAEIAKEAQDSEVLEEAVRVIEEIAKESGSEEALRQAKRAIEEIAREARDLRVEALALLAMARLYLLMVKLEQ
EEKAKEFQELLKELSERSEELIRELEEKGAASEAELARMKQQHMTAYLEAQLTAWEIESKSKIALLELQQNQLNLEL
RLMKEILRRKEKALELRKLLLAAQALVQAAAQAERQTR
>D8A_1615 Homo16meric D8 symmetric axle
(SEQ ID NO: 39)
(MG)SAEELLRRSREYLKKVKEEQERKAKEFQELLKELSERSEELIRELE[EKGAASEA]ELARMKQQHMTAYLEAQ
LTAWEIESKSKIALLELQQNQLNLELRLHEAQKRRKEKALELRKLLLAAEALVEAARQAERETR
>D8A_6043 Homo16meric D8 symmetric axle
(SEQ ID NO: 40)
(MG)SAEELLRRSREYLKKVKEEQERKAKEFQELLKELSERSEELIRELE[EKGAASEA]ELARMKQQHMTAYLEAQ
LTAWEIESKSKIALLELQQNQLNLELRARALEAHLIALAARLKVEAAKAQAAADAIRKAAEEAR
>D_1na0C3_int2_1138 Homohexameric D3 symmetric axle
(SEQ ID NO: 41)
(MG)SDEQDTLLDRMIREAAEAAKRALEAQARQQRTQSKDEAELAYLLGELAYKLGEYRIAIRAYRIALKRDPNNAE
AWYNLGNAYYKQGDYDEAIEYYQKALELDPNNAEAWYNLGNAYYKQGDYDEAIEYYQKALELDPNNAEAKQNLGNAK
QKQG(GSHHHHHH)
>D_1na0C3_int2_418 Homohexameric D3 symmetric axle
(SEQ ID NO: 42)
(MG)DHDAEEMFKRAAHASKRASKENADAAELLATAIAKDLAELAYLLGELAYKLGEYRIAIRAYRIALKRDPNNAE
AWYNLGNAYYKQGDYDEAIEYYQKALELDPNNAEAWYNLGNAYYKQGDYDEAIEYYQKALELDPNNAEAKQNLGNAK
QKQG(GSHHHHHH)
>D_1na0C3_int2_441 Homohexameric D3 symmetric axle
(SEQ ID NO: 43)
(MG)SSEAKELIEKALKNLLKIATKQAELQATIVKAQALDVAELAYLLGELAYKLGEYRIAIRAYRIALKRDPNNAE
AWYNLGNAYYKQGDYDEAIEYYQKALELDPNNAEAWYNLGNAYYKQGDYDEAIEYYQKALELDPNNAEAKQNLGNAK
QKQG(GSHHHHHH)
>D_1na0C3_int2_663 Homohexameric D3 symmetric axle
(SEQ ID NO: 44)
(MG)SEHNKDMITEALRVFEEAAEMAARAYKTLVTAQNQSVAELAYLLGELAYKLGEYRIAIRAYRIALKRDPNNAE
AWYNLGNAYYKQGDYDEAIEYYQKALELDPNNAEAWYNLGNAYYKQGDYDEAIEYYQKALELDPNNAEAKQNLGNAK
QKQG(GSHHHHHH)
>D_tj10C4_G1_678 Homooctameric D4 symmetric axle
(SEQ ID NO: 45)
(MHHHHHHGS)DECEEKARRVAEKVERLKRSGTSEDEIAEEVAREISEVIRTLKESGSSYEVICECVARIVAEIVEA
LKRSGTSAVEIAKIVARVISEVIRTLKESGSSYEVICECVARIVAEIVEALKRSGTSAAIIALIVALVISEVIRTLK
ESGSSFEVILECVIRIVLEIIEALKRSGTSEQDVMLIVMAVLLVVLATLHREDQKVNNTALAIMMEALAEAAQLAAE
AAKELKKSV
>DC4G1_1558 Homooctameric D4 symmetric axle
(SEQ ID NO: 46)
SEEEARTIAKEAATAFAKLALLQAEAFATLVKAAARVAYILGAIAYAQGEYDIAITAYQVALDLDPNNAEAWYNLGN
AYYKQGDYDEAIEYYQKALELDPNNAEAWYNLGNAYYKQGDYDEAIEYYQKALELDPNNAEAKQNLGNAKQKQG(GS
HHHHHH)
>DC4G1_178 Homooctameric D4 symmetric axle
(SEQ ID NO: 47)
(MHHHHHHGS)DECEEKARRVAEKVERLKRSGTSEDEIAEEVAREISEVIRTLKESGSSYEVICECVARIVAEIVEA
LKRSGTSAVEIAKIVARVISEVIRTLKESGSSYEVICECVARIVAEIVEALKRSGTSAAIIALIVALVISEVIRTLK
ESGSSFEVILECVIRIVLEIIEALKRSGTSEQDVMLIVMAVLLVVLATLQTEILKAINHALAVMAQALAEAAQRAAE
AAKKSATHI
>DSS_310_117 Homohexameric D3 symmetric axle
(SEQ ID NO: 48)
(MG)TLVEILARAQIESSRVNIELAREALERAKR(GSHHHHHH)
>DSSR2_1552 Homohexameric D3 symmetric axle
(SEQ ID NO: 49)
(MHHHHHHGS)SQEEESKRLVEEIAKRLKKIAEEIARAVEKLARAIIEALEVAWRSNKKIS
>SB13_1na0C3_A Homohexameric C3 symmetric axle
(SEQ ID NO: 50)
SEYEIRKALEELKAATAELKRATASLRAITEELKRLAKALAEKMYKAGNAMYRKGQYTIAIIAYTLALLADPNNAEA
WYNLGNAAYKKGEYDEAIEAYQKALELDPNNAEAWYNLGNAYYKQGDYDEAIEYYQKALELDPNNAEAKQNLGNAKQ
KQG
>SB13_1na0C3_B
(SEQ ID NO: 51)
ALVEHNRAIVEHNAIIVEHNRIIAAVLELIVRAIAHTAAELAYLLGELAYKLGEYRIAIRAYRIALKLDPNNAEAWY
NLGNAYYKQGDYDEAIEYYQKALELDPNNAEAWYNLGNAYYKQGDYDEAIEYYQKALELDPNNAEAKQNLGNAKQKQ
G

In one embodiment, any amino acid substitutions at interface residues (single underlined residues) are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In another embodiment, any amino acid substitutions at structural residues (bold font residues) are conservative amino acid substitutions.

In a further embodiment, any amino acid substitutions at residues needed for binding to small molecule (residues within squiggly brackets) are conservative amino acid substitutions.

In one embodiment, one or more loop regions are substituted or added to with any peptide domain deemed suitable for an intended use: domains that can be modified by enzymatic activity (i.e. phosphorylation), small molecule or protein binding domains, or catalytic domains. In this embodiment, the loop region may be substituted in its entirety, or 1, 2, 3, 4, 5, or all amino acid residues of the loop region may be retained when inserting the peptide domain.

In other embodiments, interface residues, structural residues, and/or residues needed for binding to small molecule are not substituted and are maintained relative to the reference polypeptide.

In another embodiment, any amino acid substitutions relative to the reference polypeptide are conservative amino acid substitutions. In one embodiment, optional amino acid residues are absent and are not considered when determining percent identity. In another embodiment, 1, 2, 3, 4, 5, 6, or more, or all of the optional amino acid residues are present and are considered when determining percent identity.

In another embodiment, the disclosure provides kits or machine assembly, comprising an axle and ring pair comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of any one or more axle and ring pair are selected from the group consisting of the following pairs (A)-(J), not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity:
(A) SEQ ID NO:5 and SEQ ID NO:6;
(B) SEQ ID NO:7 and SEQ ID NO:8;
(C) SEQ ID NO:9 and SEQ ID NO:10;
(D) SEQ ID NO:11 and SEQ ID NO:12;
(E) SEQ ID NO:13 and SEQ ID NO:14;
(F) SEQ ID NO:15 and SEQ ID NO:17;
(G) SEQ ID NO:18 and SEQ ID NO:19;
(H) SEQ ID NO:20 and SEQ ID NO:21;
(I) SEQ ID NO:22 and SEQ ID NO:23; and/or
(J) SEQ ID NO:24 and SEQ ID NO:25.

In kit embodiments, the axle and ring may be assembled or may be unassembled. In machine assembly embodiments, the axle and ring are assembled (such as by non-covalent assembly), as disclosed in the examples that follow.

In one embodiment, any amino acid substitutions at interface residues (single underlined residues) are conservative amino acid substitutions. In another embodiment, any amino acid substitutions at structural residues (bold font residues) are conservative amino acid substitutions. In a further embodiment, any amino acid substitutions at residues needed for binding to small molecule (residues within squiggly brackets) are conservative amino acid substitutions. In one embodiment, one or more loop regions are substituted or added to with any peptide domain deemed suitable for an intended use.

In other embodiments, interface residues, structural residues, and/or residues needed for binding to small molecule are not substituted. In some embodiments, optional amino acid residues are absent and are not considered when determining percent identity. In other embodiments, optional amino acid residues are present and are considered when determining percent identity. In another embodiment, any amino acid substitutions relative to the reference polypeptides are conservative amino acid substitutions.

The kit or machine assembly may comprise any other components as deemed appropriate for an intended use. In one non-limiting embodiment, the kits further comprise small molecule fuels to permit rotation of the assembled motor assembly, or small molecule suicide inhibitors that can lock mechanical rotation, as described in examples that follow.

In another aspect the disclosure provides nucleic acids encoding the polypeptides or kit/machine components of any embodiment or combination of embodiments of the disclosure. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids, expression vectors (i.e.: episomal or chromosomally integrated), non-naturally occurring polypeptides, fusion protein, or compositions disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another aspect, the present disclosure provides pharmaceutical compositions, comprising one or more polypeptides, kits, motor assemblies, nucleic acids, expression vectors, and/or host cells of the disclosure and a pharmaceutically acceptable carrier. The pharmaceutical compositions of the disclosure can be used, for example, in the methods of the disclosure described below. The pharmaceutical composition may comprise in addition to the polypeptide of the disclosure (a) a lyoprotectant; (b) a surfactant; (c) a bulking agent; (d) a tonicity adjusting agent; (e) a stabilizer; (f) a preservative and/or (g) a buffer.

The disclosure further provides methods for using the polypeptide, kit, machine, nucleic acid, expression vector, host, and/or pharmaceutical composition of any preceding claim for any suitable use as disclosed herein, including but not limited to in protein nanomachines that be genetically encoded for multicomponent self-assembly within cells or in vitro, facilitating fabrication or in vivo transfer and use in a vast range of nanodevices for medicine, material sciences or industrial bioprocesses.

In another aspect, the disclosure provides methods for designing the polypeptides of the disclosure, comprising any design methods as disclosed in the examples that follow.

### EXAMPLES

Intricate protein nanomachines in nature have evolved to process energy and information by coupling biochemical free energy to mechanical work. The design of dynamic protein mechanical systems is of great interest given their richer functionality, but while recent advances in protein design now enable the generation of increasingly sophisticated static nanostructures and assemblies(9-1 7), the complex folding and diversity of non-covalent interactions has thus far made this very challenging(18).

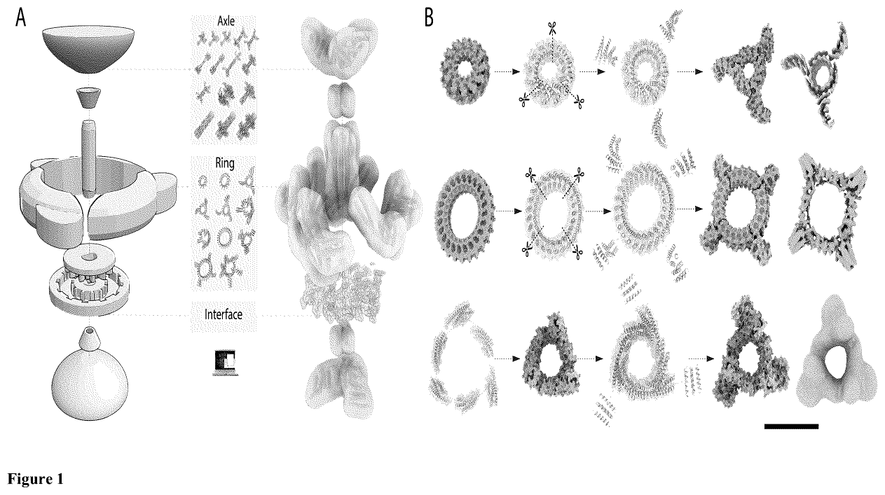

We set out to explore the design of protein mechanical systems through a first-principle, bottom-up approach that decouples operational principles from the complex evolutionary trajectory of natural nanomachines. Sampling of the folding landscape for both structural and dynamic features is computationally expensive, and hence we decided on a hierarchical design approach with steps that can be tackled in turn: (i) the de novo design of stable protein building blocks optimized for assembly into constrained mechanical systems, (ii) the directed self-assembly of these components into hetero-oligomeric complexes, (iii) the shaping of the multistate energetic landscape along mechanical degrees of freedom (DOF) and (iv) the coupling of chemical or light energy to rotation or other motion. In this paper, as a proof of concept we aim to assemble a simple machine or kinematic pair (19,20) at the nanoscale, and focus on steps i-iii to design mechanically constrained heterooligomeric protein systems that undergo brownian rotary motion. We start from a rotary machine blueprint (FIG. 1A) in which, similar to natural rotary systems, the features of the rotational energy landscape are determined by the symmetry of the interacting components, their shape complementarity and specific interactions across the interface.

### Computational Design of Protein Rotary Machine Components

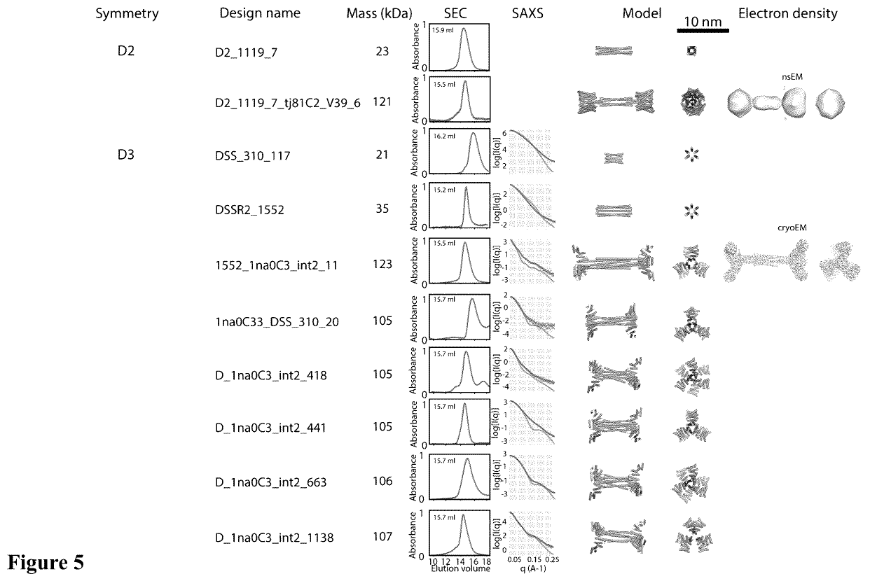

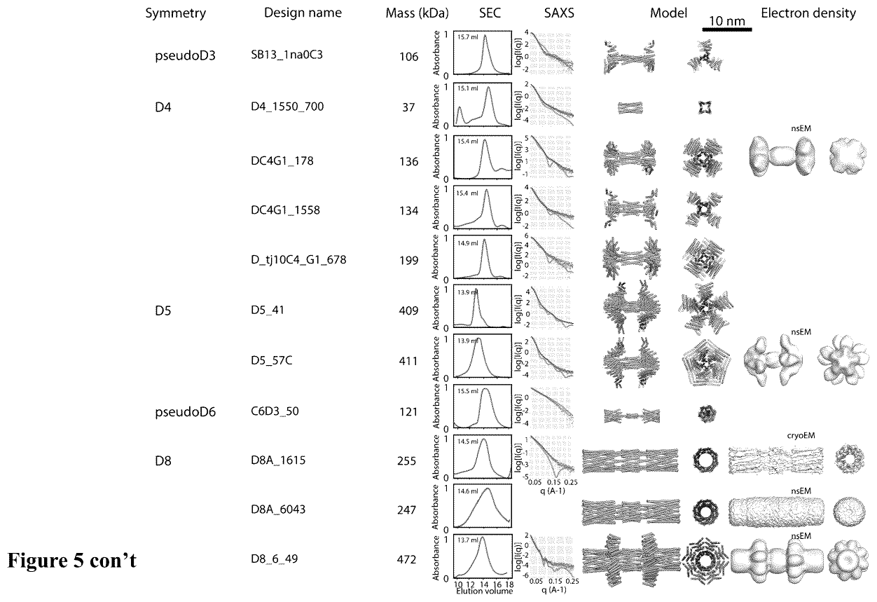

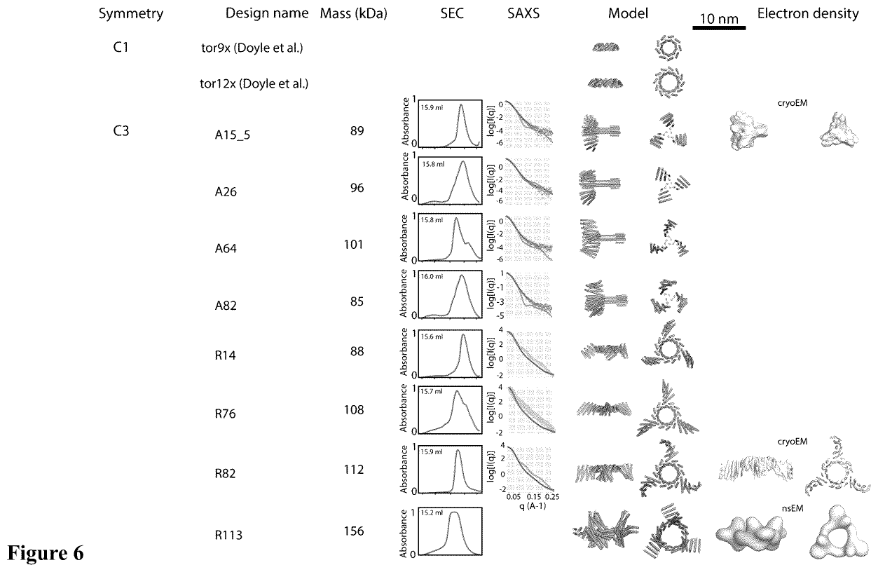

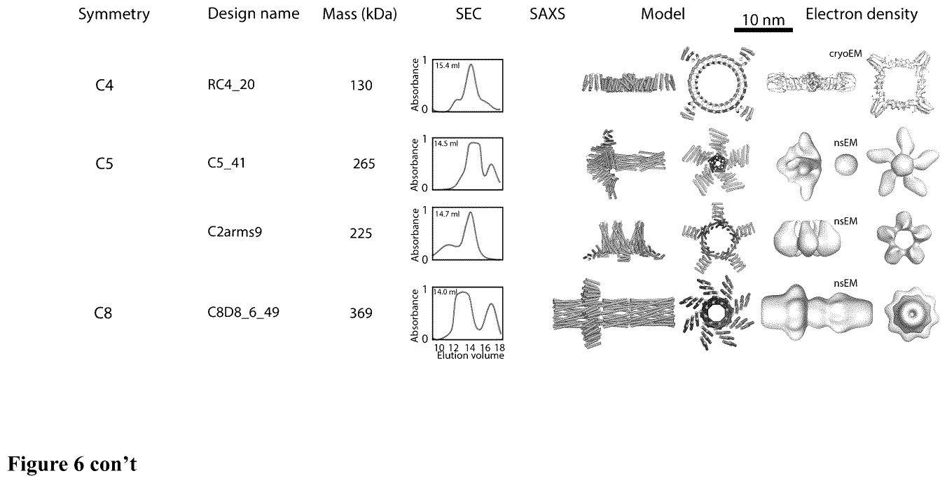

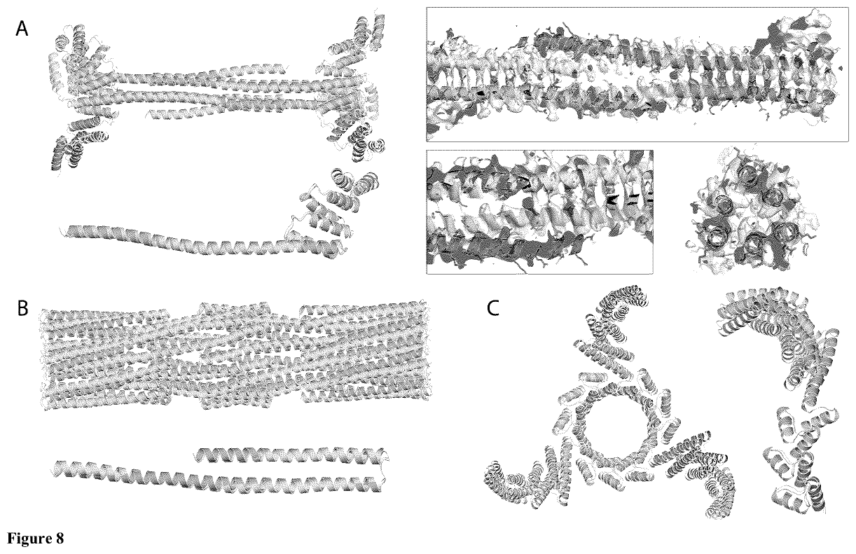

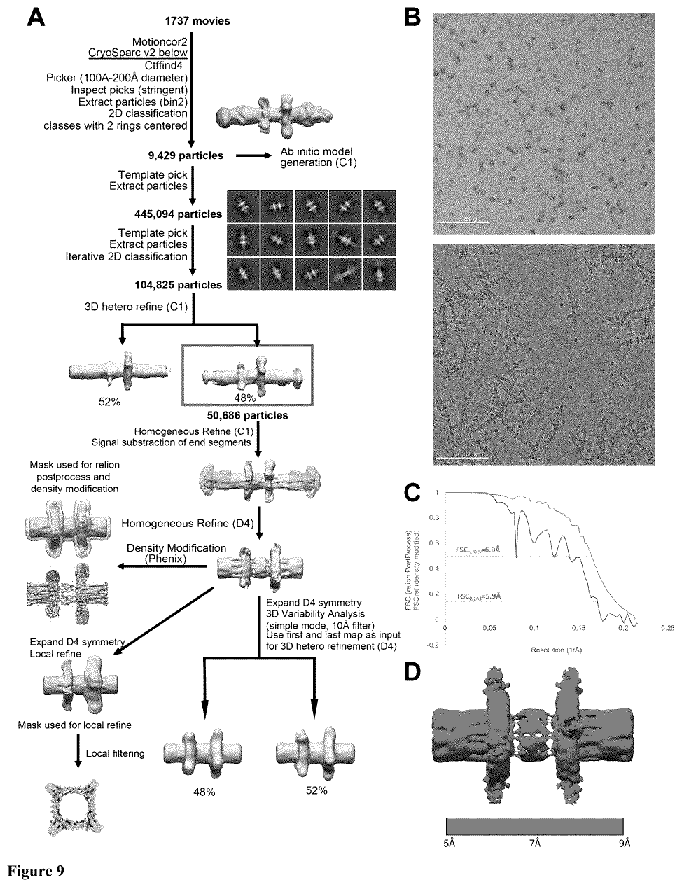

We set out to design de novo a library of stable protein components with shapes, fold and symmetry specifications suitable for integration into rotationally constrained assemblies. We first sought to design ring-like protein topologies with a range of inner diameter sizes capable of accommodating an axle-like binding partner in the center (FIG. 1B). In a first design approach, we started from de novo designed alpha-helical tandem repeat proteins (21), which were redesigned to be C1 single chain structures or symmetric C3 or C4 homooligomers. In a second approach, we used a hierarchical design procedure based on architecture-guided rigid helical fusion (12) to build C3 and C5 cyclic symmetric ring like structures by modularly assembling via rigid fusion de novo helical repeat proteins (DHRs) and helical bundle heterodimers. To facilitate experimental characterization by optical and electron microscopy, we increased the radius and total mass of the designs by fusing another set of DHRs at the outer side of the rings, generating arm-like extensions (FIGS. 1A-B). Synthetic genes encoding these designs (12xC3s, 12xC4s, 2xC5s) were synthesized and the proteins expressed in E. coli. All designed proteins were soluble after purification by nickel-nitrilotriacetic acid (Ni-NTA) and ˜23% (6/26) had appropriate monodisperse size exclusion chromatography (SEC) profiles that matched the expected theoretical elution profile for the oligomerization state. These designs were further examined using small-angle X-ray scattering (SAXS) (22,23), negative stain electron microscopy or cryoelectron microscopy (cryoEM) (FIG. 5, fig S2). For the R82 C3 ring, SAXS data was consistent with the computational model and we were able to determine using cryoEM a 6.5 Å 3D reconstruction which was very close to the design model (FIG. 1B, FIGS. 6-9, Table S1). Additional designs of the same topology (R14 and R76) were characterized by SAXS and showed similar profiles, and hence likely have the same oligomeric state and overall structure (FIG. 6). A C3 ring with larger inner diameter and different topology (R113) was characterized using negative stained EM, yielding a low resolution 3D reconstruction consistent with the design model (FIG. 1B, FIG. 6). For a C4 design highly expressed in E. coli, we obtained a ˜5.9 Å cryo electron density map revealing a structure nearly identical to the design model (FIG. 1B, FIG. 6, FIG. 9, Table S1). Negative stain EM of a C5 ring yielded a low resolution 3D map consistent with the design model (FIG. 6).

TABLE S1
Cryo-EM data collection and Refinement statistics

| | C3-C3 | | D3 axle | | D3-C5 | | D3-C3 | | D8-C4 | |
|---|---|---|---|---|---|---|---|---|---|---|
| Number of | 6,072 | | 3,512 | | 9,364 | | 5,084 | | 1,737 | |
| micrographs | | | | | | | | | | |
| Nominal | 130,000X | | | | | | | | 36,000X | |
| magnification | | | | | | | | | | |
| Voltage | 300 kV | | | | | | | | 200 | kV |
| Electron Fluence | 90 e⁻/Å² | | | | | | | | 65 | e⁻/Å² |
| Pixel size | 1.05 Å | | | | | | | | 1.16 | Å |
| Defocus range | −1.7 | | −1.5 | | −1.3 | | −0.5 | | −1.4 | |
| | to −2.8 μm | | to −2.4 μm | | to −2.5 μm | | to −3.8 μm | | to −4.2 μm | |
| EMDB ID | | | | | | | | | | |
| Map resolution | 6.5 | Å | 6.2 | Å | 8 | Å | 10.2 | Å | 7.2 | Å |

| 0.143 FSC | | | | | | | |
|---|---|---|---|---|---|---|---|
| Density Modified | n/a | 4.2 | Å | n/a | n/a | 7.0 | Å |

| Resolution 0.5Ref | | | | | |
|---|---|---|---|---|---|
| Symmetry | C3 | D3 | C1 & | C3 | D4 |
| Imposed | | | D3 | | |
| Number of | 25,437 | 33,479 | 73,042 (D3) | 16,244 | 50,686 |
| particles | | | 57,764 (C1) | | |

Refinement

| | | | | | | | | | | |
|---|---|---|---|---|---|---|---|---|---|---|
| Map Sharpening B | −330 | Å² | −284 | Å² | −471 | Å² | −503 | Å² | −430 | Å² |

Factor

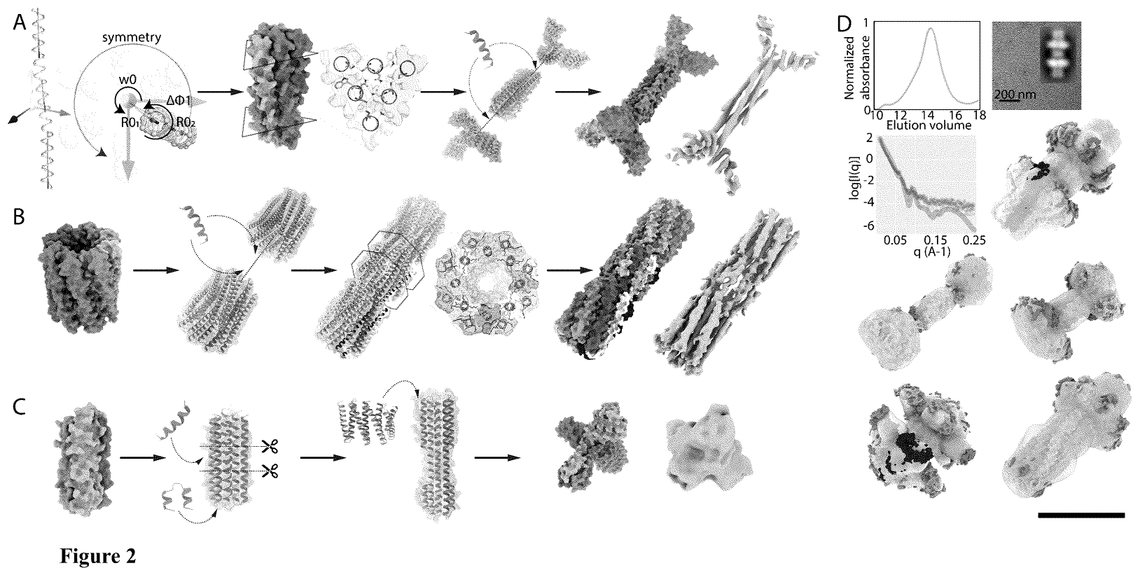

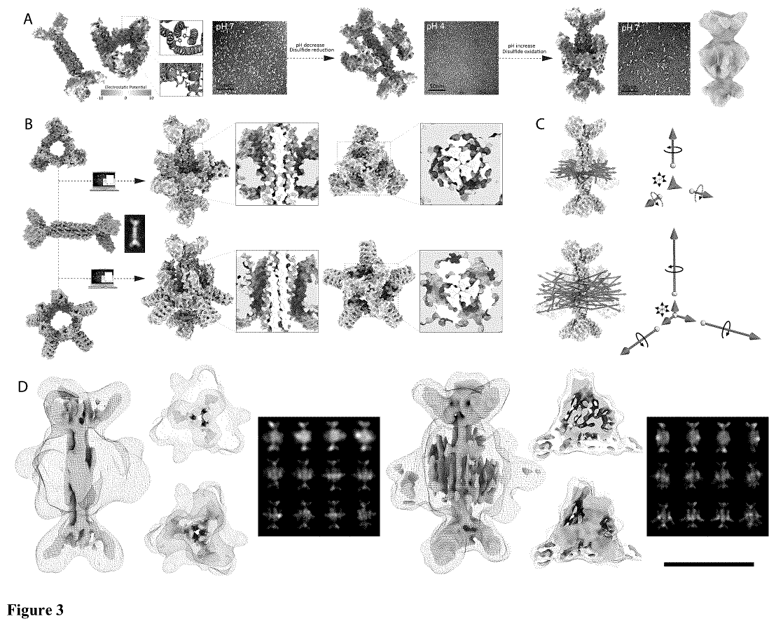

We next sought to design high aspect ratio protein folds, or axles, onto which the ring-like designed protein could be threaded. In a first approach, single helix protein backbones were parametrically generated, and then D2, D3 or D4 dihedral symmetry was imposed to produce self-assembling dihedral homooligomers consisting of interdigitated single helices (FIG. 2A). Two helices were placed roughly colinearly along the z axis but at different distances from it, their superhelical parameters were sampled using the Crick-generating equations (24), and those for which imposition of dihedral symmetry generated closely packed structures were connected with a linking helix (see “Computational design methods” in the supplementary materials). Rosetta™ HBNet (25) was then used to install hydrogen bond networks with buried polar residues between the helices (4, 6, or 8 for a D2, D3 and D4 respectively) to generate homooligomeric interfaces with the high level of specificity needed for dihedral assembly. The sequence of the rest of the homooligomer (surface residues and the hydrophobic contacts surrounding the networks) was then optimized while keeping the networks constrained during Rosetta™ Design as described previously (25). Last, in order to increase the total mass, diversify the shape as well as increase the modularity of axles, each helix of the best-scoring designed dihedral homooligomers was connected at either the C or N terminus to an outer helix belonging to de novo cyclic homooligomer wheels of matching symmetry (i.e. Cn ->Dn), through a short helical fragment sampled and designed using Rosetta™ Remodel, to finally produce full axle homooligomers. In a second approach, de novo cyclic homooligomers were selected (15) and Rosetta™ BlueprintBuilder (26) was used to generate interdigitated helical fragments of varying length and topology which were computationally extensively sampled at the N or C terminus in order to direct the assembly into dihedral homooligomers (FIG. 2B, see “Computational design methods” in the supplementary materials). In a third approach, cyclic homotrimer backbones consisting of helical hairpin monomer topologies with inner and outer helices that were previously parametrically generated t2.5) were circularly permuted by re-looping terminis using the Rosetta™ ConnectChainsMover, placing terminis in the middle of the outer helices, and elongating inner helix heptad repeats to generate C3 homooligomers which 3 inner helix form an accessible surface for further rotary machine design (FIG. 2C).

Synthetic genes encoding axle designs generated from the three approaches (12xC3s, 12xC5s, 12xC8s, 6xD2s, 12xD3s, 6xD4s, 6xD5s, 12xD8s) were obtained and the proteins were expressed in E. coli. The designed proteins that were well-expressed, soluble, and readily purified by Ni-NTA affinity chromatography were further purified on SEC. ˜40% (37.5% (6/16), 43% (14/32) and 33% (4/12) success rates for the first, second and third approach respectively) had appropriate monodisperse SEC chromatograms that matched the expected theoretical elution profile for the oligomerization state (FIG. 2D, FIGS. 5-6). These designs were then further examined using either SAXS, negative stain electron microscopy, cryoEM or a combination of techniques (FIGS. 5-6). Details of the methods, as well as scripts for carrying out the design calculations, are provided in the supplementary materials.

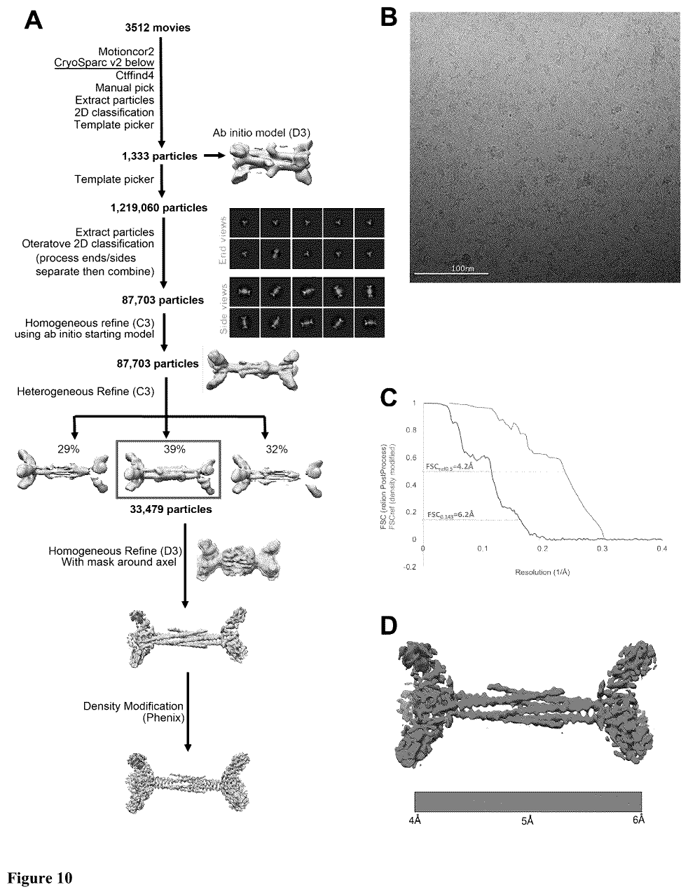

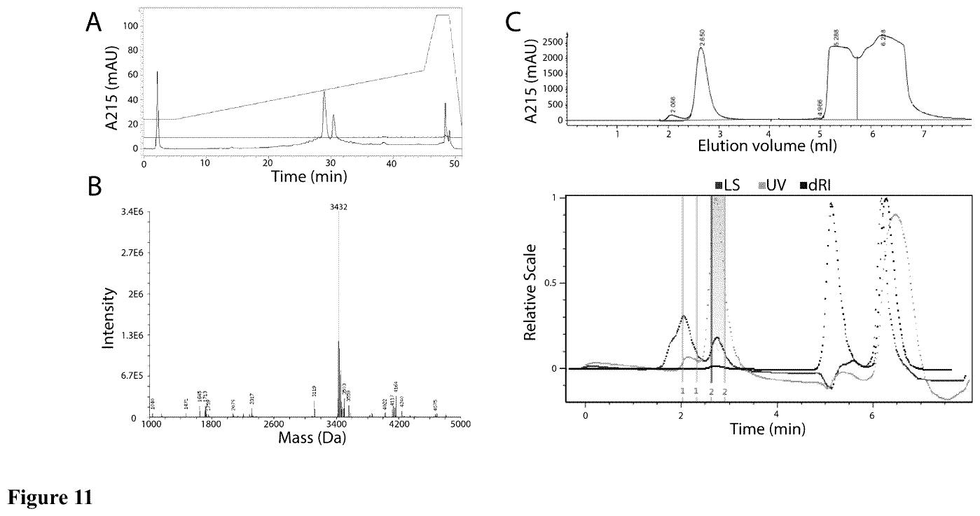

The first approach generated D2, D3 and D4 axle-like structures with folds featuring interdigitated helices with extended hydrogen bond networks. We obtained a 4.2A 3D reconstruction of a D3 axle (_1na0C3_int2_11) which showed close agreement with the design model topology. While the backbone was nearly identical to the design model, the side-chains could be partially elucidated (FIG. 2B, FIG. 5, FIG. 9, FIG. 10). SAXS data also showed overall good agreement with the design (FIG. 5). SAXS and SEC revealed that the middle homohexameric 50 residues long single helices (without appended DHR wheel arms) could be solubly expressed and self-assembled into the correct oligomeric state (DSSR2_1552) (FIG. 5). Another D3 design consisting of 36 residue long single helices was produced via chemical peptide synthesis and assembled into a homohexamer (DSS_310_117, FIG. 5, FIG. 11), while its fusion to C3 wheels generated a bigger D3 oligomer as designed (1na0C3_DSS310_20, FIG. 5). A D4 peptide homo-oligomer designed using the same approach (D4_1550_700) had SEC and SAXS spectra indicating the designed correct oligomeric state (FIG. 5). Negative stain EM of a D2 design (D2_1119_7_tj81C2_V39_6) yielded a low resolution 3D reconstruction with the overall features of the design model (FIG. 2D, FIG. 5). The corresponding central 50 residue D2 peptide (D2_1119_7) again expressed solubly and could be purified in the correct oligomeric state (FIG. 5).

The second approach generated D3, D4, D5 and D8 axle-like structures with folds featuring interdigitated helices with internal cavities for D5 and D8 (in these cases each central helix only forms contacts with two neighboring ones) (FIG. 2B). We obtained a ˜5.9 Å electron density map of a D8 design (D8A_1615) revealing a backbone structure nearly identical to the design model (FIG. 2B, FIG. 5, FIGS. 9-10). This cylinder-shaped homodecahexamer has a previously unobserved fold, with a large central cavity with an end-to-end pore-like feature, contains a nearly straight helix spanning 84 residues and has opposing N and C termini close to its center (FIG. 2B, FIG. 5). Negative stain EM on additional designs: two D8s (D8A_6043 and D8_6_49), one D5 (D5_57C) and one D4 (DC4G1_178), yielded low resolution 3D reconstructions with the features of the design models (FIG. 2D, FIGS. 5-6). We converted several of these designs from dihedral to cyclic symmetry by connecting N and C termini, and two such designs, one C5 (C5_41) and one C8 (C8D8_6_49), yielded EM reconstructions with good agreement with the design model (FIG. 2D, FIGS. 5-6). Other designs (six D3 s, two D4s and one D5s) for which EM data was not obtained were characterized by SAXS and showed similar profiles, which were consistent with the correct oligomeric state and overall structural features (FIG. 2D, FIGS. 5-6).

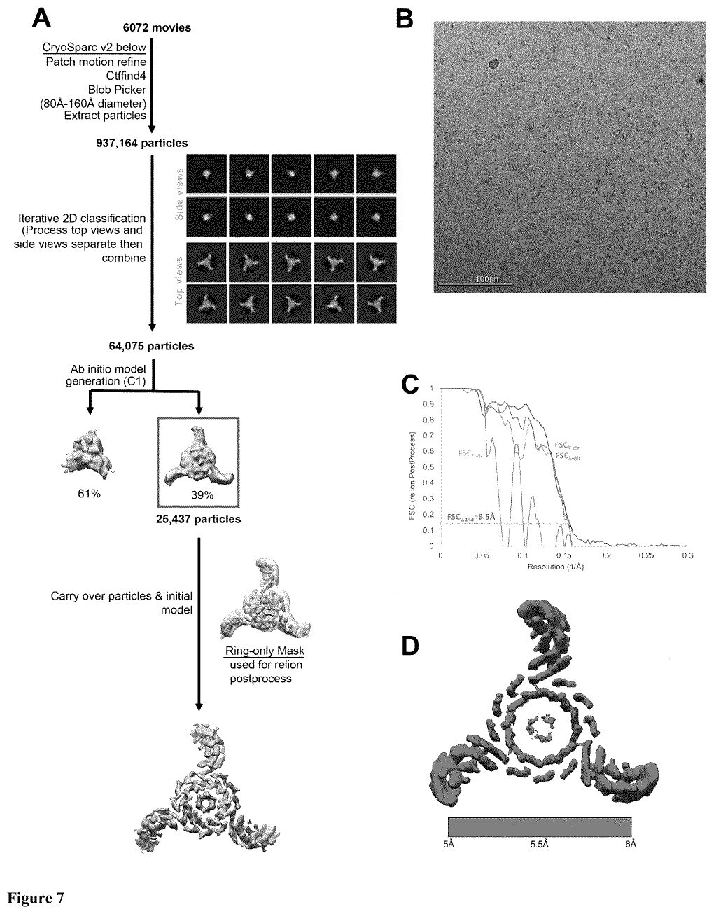

The third approach yielded four C3 axles with folds of smaller aspect ratio and overall size, containing a large wheel-like DHR feature at one end, a narrow central three helix section and a six helix section at the other end. In all cases, the SAXS profiles together with SEC traces suggested that the correct oligomerization state was realized in solution. For design A15.5 we obtained a low resolution cryoEM map that recapitulated the general features of the design model, with prominent C3 symmetric DHR extremities and opposing prism-like extensions (FIG. 2C, FIGS. 6-7).

### Design of Axle-Ring Assemblies

We next sought to assemble diverse axle-ring assemblies to explore the correspondence between the symmetry and energy landscape of the interface and the mechanical properties. The first challenge was to direct the self-assembly in solution of the ring around the axle by designing energetically favorable interactions, while maintaining some rotational freedom. We first sought to do this by designing assemblies with low residue interaction specificity, loose interface packing, as well as non-obligatory symmetry mismatched interactions between axle and ring restricting only parts of the assembly to form tight contacts (i.e. the full interface is never fully satisfied). To achieve these properties, we initially focused on electrostatic interactions between ring and axle which are longer range and less dependent on shape matching than the hydrophobic interactions generally utilized in protein design. To prevent potential disassembly at low concentrations, we aimed to kinetically trap the ring around the axle by installing disulfide bonds at the ring subunit-subunit interfaces. Further, to gain stepwise control on the in vitro assembly process, we introduced buried histidine mediated hydrogen bond networks at the ring asymmetric unit interfaces to enable pH controlled ring assembly (FIG. 3A, see “Experimental methods” in the supplementary materials).

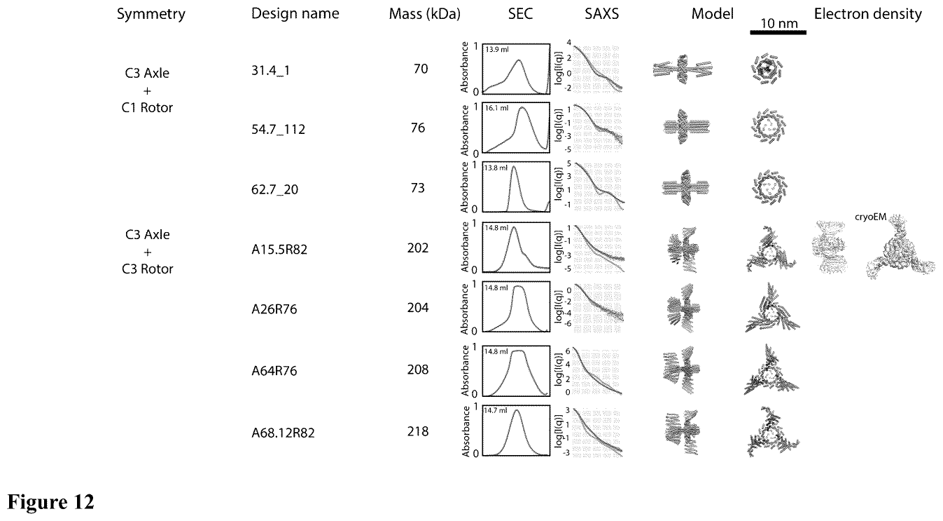

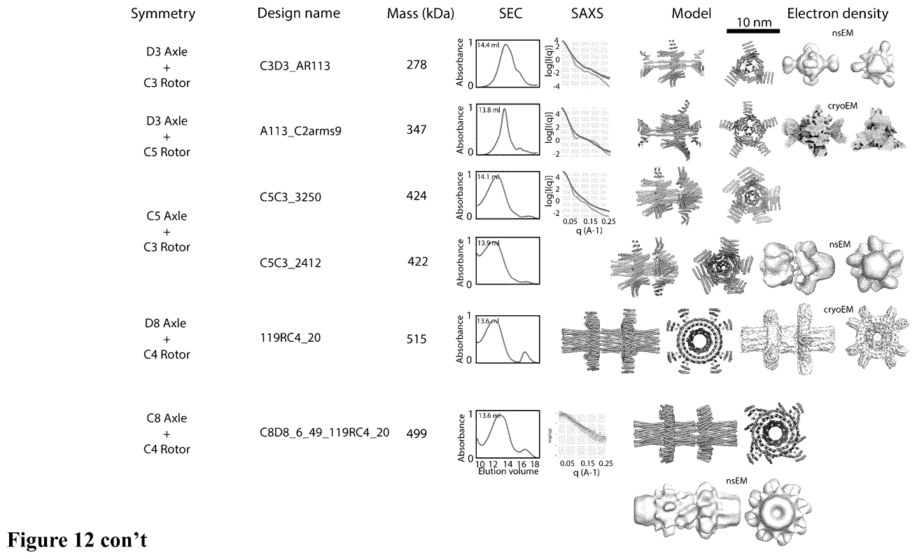

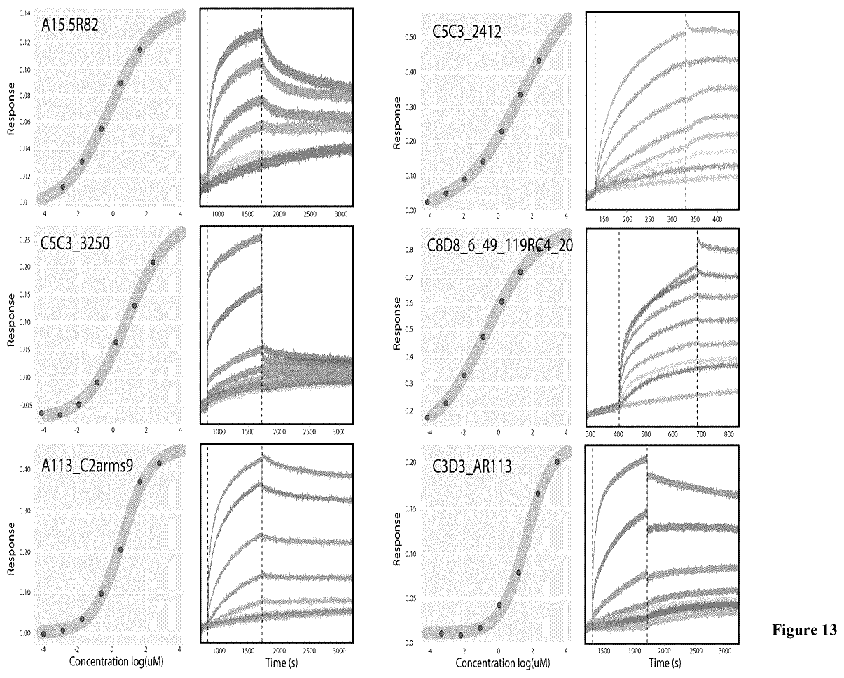

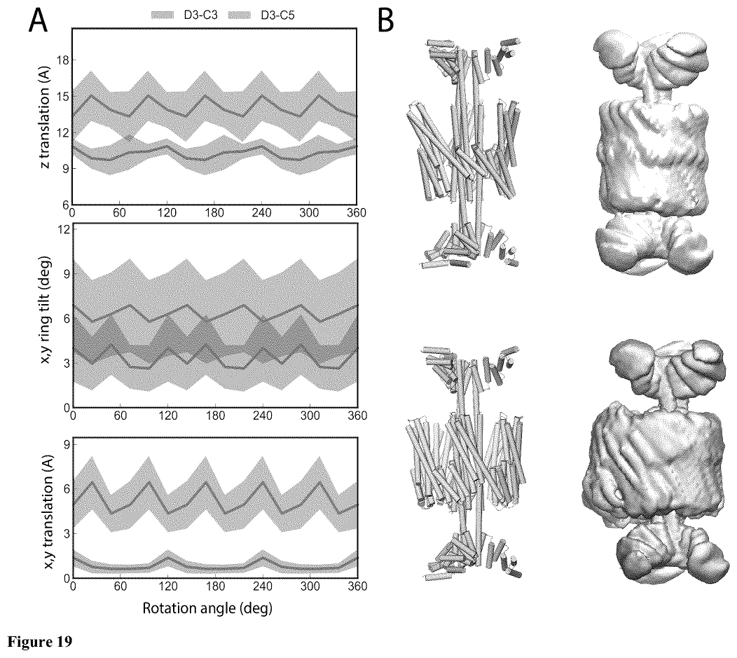

We tested this approach by selecting three of the machine components described above—a D3 axle, a C3 ring and a C5 ring—and constructing ring-axle rotary machine assemblies with D3-C3 and D3-C5 symmetries (design A113_C2ams9 and C3D3_AR113 respectively, FIG. 3B, FIG. 12). Using PyRosetta™ (27), we threaded axles and rings together by sampling rotational and translational DOF, and designed complementary electrostatic interacting surfaces excluding positively charged residue identities on the axle (Lysine and Arginine) and negatively charged residues (Aspartate and Glutamate) on the ring. Due to the shape complementarity between the internal diameter of the rings and the axle thickness, the interface is tighter for the D3-C3, constraining the ring midway on the axle, and loose for the D3-C5 where the ring can diffuse along multiple DOF, thus resulting in different mechanical constraints: the D3-C3 is only allowed to rotate along the main symmetry axis, while the D3-C5 ring can rotate along x, y and z, as well as translate in z and y (FIGS. 3B-C, FIG. 19). Synthetic genes encoding one axle and 2 ring designs were obtained and the proteins were separately expressed in E. coli and purified by Ni-NTA affinity chromatography and SEC, which indicated that the surface redesign did not affect the solubility or homo-oligomerization process (FIGS. 5-6). Following stoichiometric mixing of the designed D3 axle and C3 ring, EM analysis showed a collection of assembled and isolated axle and ring molecules (FIG. 3A, left panel). After dropping the pH and reducing the disulfide, the particles appeared as a mixture of opened, linear and hard to distinguish particles (FIG. 3A, middle panel). After restoring the pH under oxidizing conditions, the particles appeared fully assembled by EM (FIG. 3A, right panel). Using biolayer interferometry assays we found that the ring and axle associated rapidly with a Kd in the micromolar range (FIG. 13). Similar results were obtained with D3-C5 rotary assemblies, and SEC profiles and SAXS spectra were in agreement with the design model in both cases (FIG. 12).

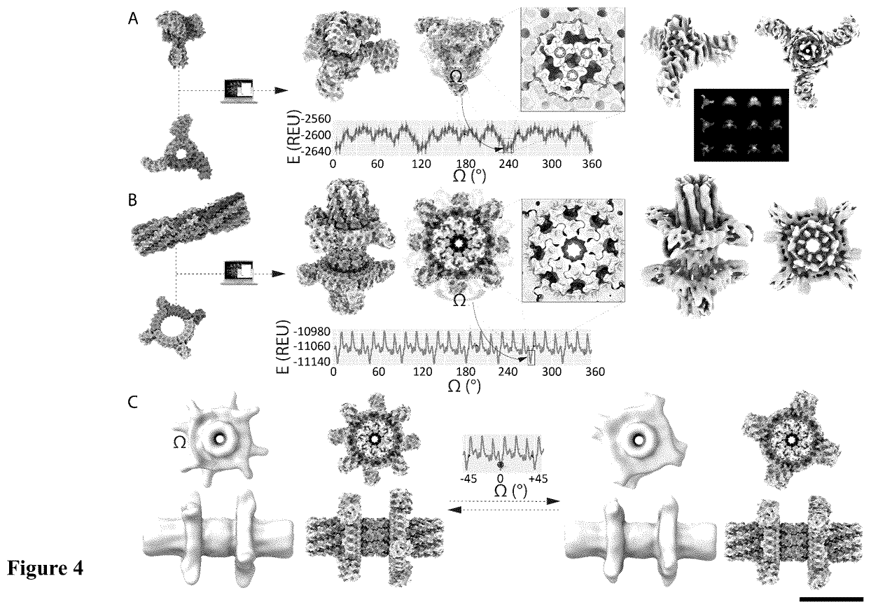

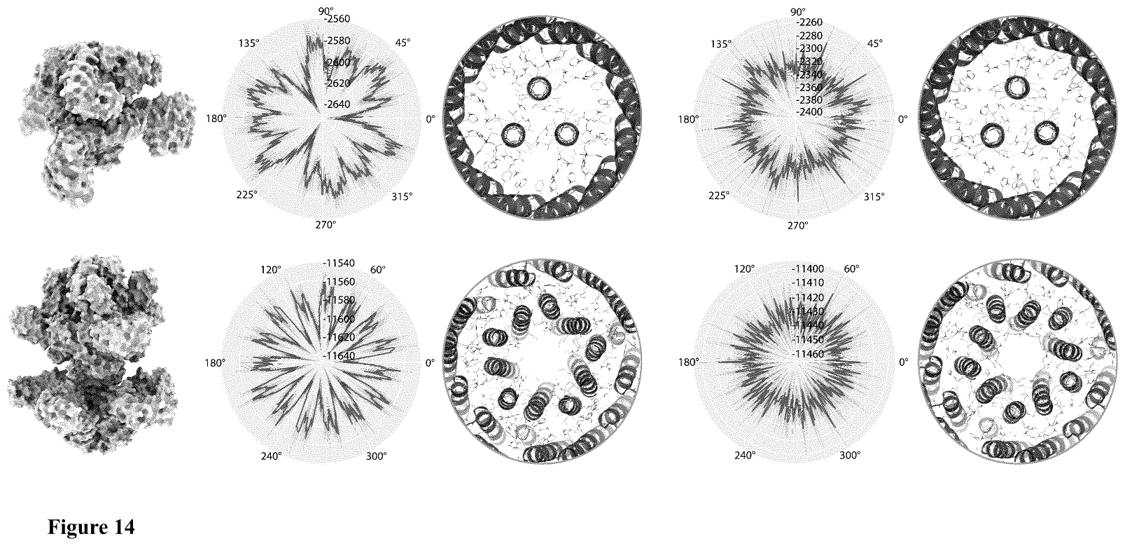

We next experimented with the design of shape complementary axle and ring components, reasoning that this would enable more precise control of the rotational energy landscape by leveraging the ability to design tightly packed interfaces and hydrogen-bond networks mediated specificity (25). We designed four axle-ring assemblies using this approach: a fully C3 symmetric assembly consisting of a C3 axle and a C3 ring (C3-C3, A15.5R82), a symmetry mismatched assembly consisting of a D8 axle around which two C4 rings are assembled (D8-C4, 119RC4_20), a symmetry mismatched rotor consisting of C5 axle and C3 ring (C5-C3_2412 and C5C3_3250), as well as a C8-C4 rotor corresponding to a circular permutation version of the D8-C4 (C8D8_6_49_119RC4_20) (FIG. 4A, FIG. 4B, FIG. 12). The symmetry matching of the ring and axle in the C3-C3 rotor differs from the mismatching in other assemblies, and the two ring D8-C4 assembly tests the incorporation of multiple coupled rotational DOF in a multicomponent system and also provides a simple way to monitor the position of rings relative to each other by experimental structural characterization, thus providing an indirect way to monitor rotation. Similarly, the DHR arms on other rotors offer direct structurally accessible monitoring of the rotation by visualizing the alignment of axle and ring arms relative to each other. These designs were generated by systematically sampling rotational and translational DOF, removing arrangements with backbone to backbone clashes (FIG. 2B, see “Computational design methods” in the supplementary materials), and then using the Rosetta™ HBnet protocol and FastDesign (28) to optimize the interface energy. Each interface design trajectory generates widely different periodic energy landscapes according to interface metrics and design specifications (FIG. 14). In the case of the D8-C4, C5-C3 and C8-C4 designs, since the symmetry of the ring is internally mismatched to the axle, we used a quasisymmetric design protocol (see “Computational design methods” in the supplementary materials). The C4 ring, which is internally C24 symmetric due to the repeated nature of sequences from which it is built, can accommodate the symmetry of D8 or C8 axles since 24 is a multiple of 8, which allows pairing of interactions at the interface while maintaining overall C4 symmetry. In contrast, the C5-C3 arrangement has broken symmetry with a resulting energy landscape with 15 energy minima, with periodicities reflecting the constituent C5 and C3 symmetries (FIG. 14). This design approach generated shape complementary axle-ring interfaces with an overall cogwheel topology.

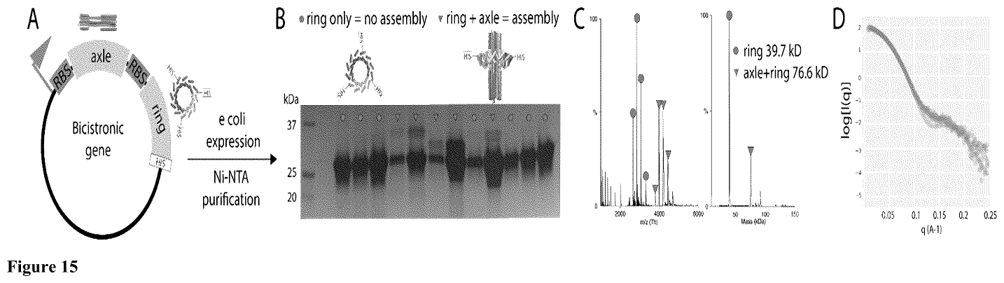

Designs with each of the four symmetries were screened for assembly by expressing ring and axle pairs bicistronically and carrying out Ni-NTA purifications relying on a single HIS tag on the ring component (FIG. 15A). ˜50% (6/12) of C3-C3 designs appeared to express solubly and could be pulled down by the purification process, suggesting that the two components assembled in cells (FIG. 15B), and one design (54.7.112, FIG. 12) was further selected for further characterization. The SEC profile in combination with native mass spectrometry indicated an oligomeric state corresponding to the designed assembly, and SAXS data collected on the protein showed good agreement with the design model (FIG. 12, FIGS. 15C-D). Using biolayer interferometry we analysed the capacity of the designed axle and ring to assemble in vitro into the full rotor, and found that this system showed rapid assembly kinetics with a Kd in the micromolar range (FIG. 13). Twelve D8-C4 designs were likewise screened for in vitro assembly by isolating axle and rings individually by Ni-NTA purifications, and then assayed for assembly by mixing components in stoechiometric fashion. These mixtures were then further SEC purified and the oligomeric assembly state could thus be assessed in addition to SAXS validation, indicating that some of these rotors could self-assemble in vitro, while EM data indicated that the rotors were assembling as designed (FIG. 12). Two out of twelve C5-C3 and one out of six C8-C4 designs tested likewise assembled into axle-ring systems based on SEC chromatograms, and SAXS data, biolayer interferometry binding kinetics and negative stain EM data were consistent with assembly (FIG. 12, FIG. 13).

### Population of Multiple Rotational States

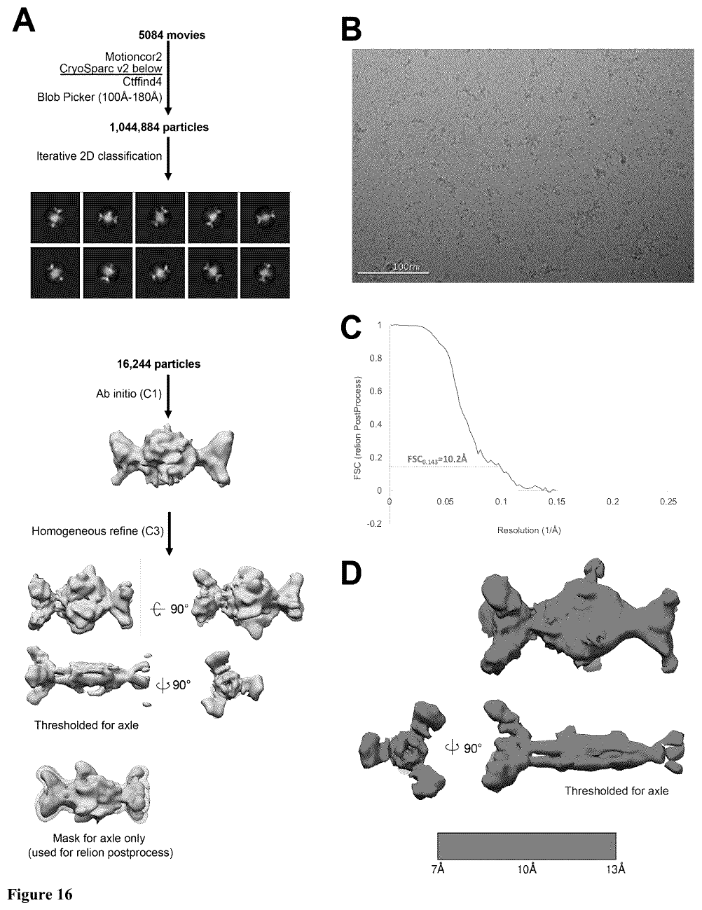

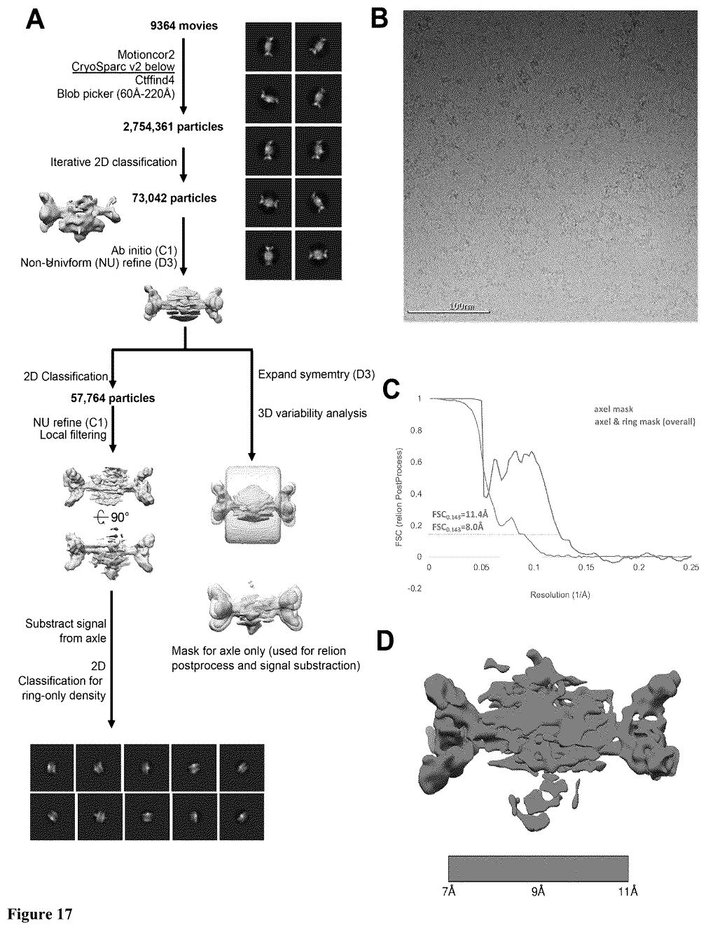

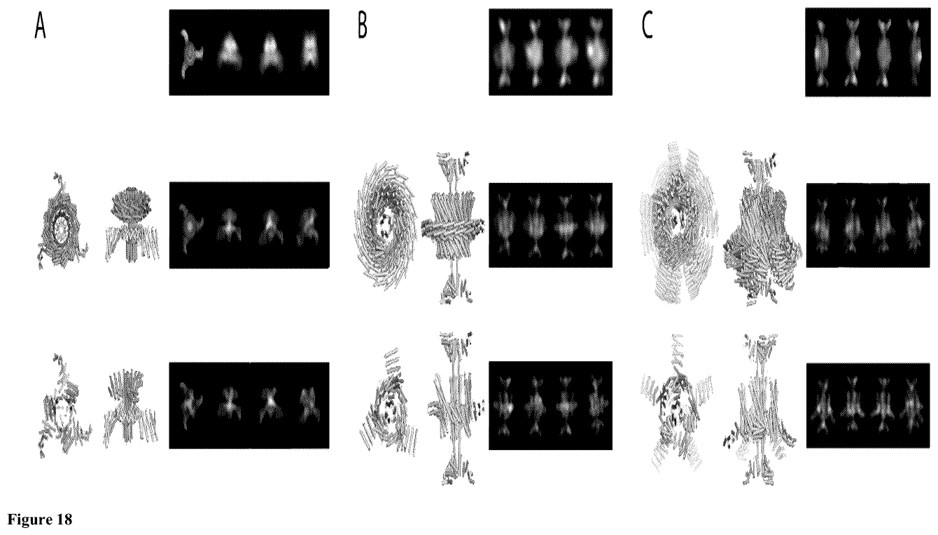

To map the rotational landscape at the single molecule level, we subjected one design from each symmetry class to single particle cryoEM examination. For D3-C3 and D3-05, we obtained 2D class averages from the collected data that clearly resembled predicted projection maps, and 3D reconstructions in close agreement with the overall design model topology and designed hetero-oligomeric state (FIG. 3D, FIGS. 16-17, Table S1). For both designs, the D3 axle was clearly visible and we obtained a high resolution structure nearly identical to the design model. We were able to obtain a high resolution 3D reconstruction map for the D3-C3 rotor assembly, which showed a clear density of the ring sitting in the middle of the axle and recapitulating the C3 ring arms extension, either after processing in C1, C3 or D3 mode (FIG. 16). The ring of the D3-C5 design also showed clear density but its resolution could not be further improved as the secondary structure placement relative to the axle were variable, likely due to motion of ring and axle along the multiple DOFs (FIG. 17). Cryosparc 3D variability analysis (29) suggested that the helical features corresponding to the ring can populate variable positions around the axle according to rotational DOFs only for D3-C3, and translational and rotational DOFs for D3-C5 (FIGS. 3B-C). This is also evident from visual inspection of the cryoEM 3D reconstruction: the ring arms populate multiple positions along the rotational axis (FIG. 3D). Explicit modelling of rotational variability along the designed DOFs was necessary to produce theoretical projections closely resembling the experimental 2D class averages (FIG. 3D, FIG. 18). Molecular dynamics simulations (MD) recapitulated the intended internal rotary motion between ring and axle, with the D3-C5 rotary machine showing increased displacement along allowed DOFs compared to D3-C3 (FIG. 3C, FIG. 19). Taken together, the cryoEM data and molecular dynamics simulations are consistent with the design goal of constrained internal rotation.

Single particle cryoEM analysis of a C3-C3 assembly yielded 2D class averages with the axle and ring clearly visible. We were able to generate a 3D reconstruction with a resolution of 6.5 Å, which yielded an electron density map similar to the design model (FIG. 4A, FIG. 7, FIG. 12, Table S1). However, the high orientation bias of the particle in ice considerably limited the resolution of the structure by preventing the obtention of side views. We hypothesize that the diffuse density of the axle in the middle of the clear ring in top view class averages could be attributed to rotational diffusion (FIG. 4A, FIG. 7). This appeared evident after explicitly modeling rotational variability along the designed DOF, which produced theoretical averages closely resembling the experimental data (FIG. 4A, FIG. 18). This is consistent with the designed smooth energy landscape with 3 energy minima at a 60° rotation distance and 9 other 30° spaced degenerate alternative wells separated by energy barriers.

The predicted energy landscape of the D8-C4 design is quite rugged, with a total amplitude of 151.7 REU with 8 steep wells spaced 45° stepwise along the rotational axis corresponding to the high symmetry of the interface. We obtained a cryoEM map of ˜5.9 Å resolution very close to the design model (FIG. 4A, FIG. 19, Table S1). 3D variability analysis calculations using Cryosparc software(30) showed that the experimental structural data could be clustered in two nearly equiprobable states which corresponded to two rotational states of one ring relative to the other, corresponding to pronounced energy-minima with 45° steps along the rotational axis consistent with the in silico designed energy landscape. There are two clearly identifiable structures in which the ring arms are either aligned or offset, as in the eclipsed and staggered arrangements of ethane (FIG. 4C, FIG. 9). While cryoEM provides a frozen snapshot of rotational bins, this data shows that the system can assemble and sample mechanical rotational bins according to the design specifications. Taken together, these results suggest that the explicit side-chain interaction design reduces the degeneracy of rotational states observed with purely electrostatic interactions.

### Conclusions

Our proof of concept rotary machine assemblies demonstrate that protein nanostructures with internal mechanical constraints can now be designed. The hetero-oligomers topologies we created do not exist in nature nor have such synthetic systems been designed previously, and provide insights towards the design of more complex protein nanomachines. First, systematic and accurate de novo design according to machine components specification (FIG. 1, FIG. 2), coupled with computational sculpting of the interface between parts can be used to simultaneously promote self-assembly and constrain motion along internal degrees of freedom. Second, the shape and periodicity of the resulting rotational energy landscape is determined by the symmetry of components, the shape complementarity of the interface, and the balance between hydrophobic packing and conformationally promiscuous electrostatic interactions (FIG. 3, FIGS. 4A-C). Symmetry mismatch tend to generate assemblies with larger numbers of rotational energy minima than symmetry matched ones, and explicit design of close sidechain packing across the interface results in deeper minima and higher barriers than non-specific interactions (FIG. 3, FIG. 4, FIG. 14). In general, the surface area of the interface between axle and ring scales with the number of subunits in the symmetry, resulting in a larger energetic dynamic range accessible for design (FIG. 14). The combination of the structural variability apparent in the cryoEM data of D3-C3, D3-C5 and C3-C3 designs (FIG. 3D, FIG. 4, FIG. 7, FIGS. 16-18), the MD simulations (FIG. 3C, FIG. 19), and the discrete states observed for the D8-C4 design (FIG. 4C, FIG. 9), suggests that these assemblies sample multiple rotational states. Time-resolved characterization of the internal motion at the single molecule level will reveal how the ability to computationally shape rotational energy landscapes can be used to control Brownian dynamics.

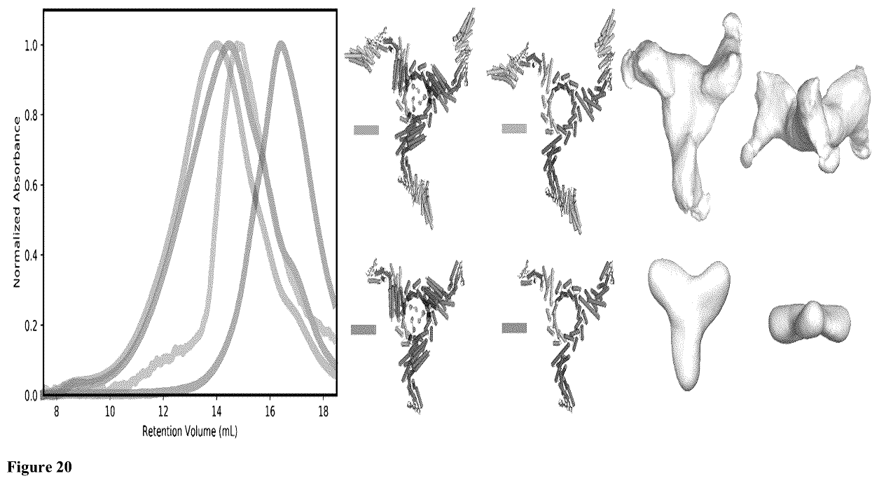

The internal periodic but asymmetric rotational energy landscapes of our designed rotary machine assemblies provide one of two needed elements for a directional motor. An energy harvesting process to break detailed balance and transfer the system into an excited state remains to be designed: for example the interface between machine components can be designed for binding and catalysis of small molecule fuels (19). Symmetry mismatch, which plays a crucial role in torque generation in natural motors (31-37), can be leveraged for the design of synthetic protein motors. Modular assembly could lead to compound machines for advanced operation or integration within nanomaterials. In this direction, we recently designed modular rotor complexes with reversible heterodimer extensions binding components of the rotor (FIG. 20). Our protein nanomachines can be genetically encoded for multicomponent self-assembly within cells (FIG. 15) or in vitro (FIG. 14), facilitating fabrication or in vivo transfer and use. Taken together, these approaches can be used in a vast range of nanodevices for medicine, material sciences or industrial bioprocesses. More fundamentally, de novo design provides a bottom-up platform to explore the critical principles and mechanisms underlying nanomachine function that complements long standing more descriptive studies of the elaborate molecular machines produced by natural evolution.

### REFERENCES AND NOTES

1. Junge, W. & Nelson, N. ATP Synthase. Annu. Rev. Biochem. 84, 631-657 (2015).
2. Feynman, R. P. There's Plenty of Room at the Bottom. in vol. 23 (5) 22-36 (California Institute of Technology Journal of Engineering and Science, 1959).
3. Zhang, L., Marcos, V. & Leigh, D. A. Molecular machines with bio-inspired mechanisms. Proc. Natl. Acad. Sci. 115, 9397-9404 (2018).
4. Drexler, K. Building molecular machine systems. Trends Biotechnol. 17, 5-7 (1999).
5. Feringa, B. L. The Art of Building Small: From Molecular Switches to Molecular Motors. J. Org. Chem. 72, 6635-6652 (2007).
6. Sauvage, J.-P. From Chemical Topology to Molecular Machines (Nobel Lecture). Angew. Chem. Int. Ed. 56, 11080-11093 (2017).
7. Cheng, C. & Stoddart, J. F. Wholly Synthetic Molecular Machines. ChemPhysChem 17, 1780-1793 (2016).
8. Ramezani, H. & Dietz, H. Building machines with DNA molecules. Nat. Rev. Genet. 21, 5-26 (2020).
9. Baker, D. What has de novo protein design taught us about protein folding and biophysics? Protein Sci. 28, 678-683 (2019).
10. Butterfield, G. L. et al. Evolution of a designed protein assembly encapsulating its own RNA genome. Nature 552, 415-420 (2017).
11. Chen, Z. et al. De novo design of protein logic gates. Science 368, 78-84 (2020).
12. Hsia, Y. et al. Hierarchical design of multi-scale protein complexes by combinatorial assembly of oligomeric helical bundle and repeat protein building blocks. biorxiv.org/lookup/doi/10.1101/2020.07.27.221333 (2020) doi:10.1101/2020.07.27.221333.
13. Langan, R. A. et al. De novo design of bioactive protein switches. Nature 572, 205-210 (2019).
14. Ueda, G. et al. Tailored design of protein nanoparticle scaffolds for multivalent presentation of viral glycoprotein antigens. eLife 9, e57659 (2020).
15. Xu, C. et al. Computational design of transmembrane pores. Nature 585, 129-134 (2020).
16. Divine, R. et al. Designed proteins assemble antibodies into modular nanocages. Science 372, eabd9994 (2021).
17. Ben-Sasson, A. J. et al. Design of biologically active binary protein 2D materials. Nature 589, 468-473 (2021).
18. Kuhlman, B. & Bradley, P. Advances in protein structure prediction and design. Nat. Rev. Mol. Cell Biol. 20, 681-697 (2019).
19. Flechsig, H. & Mikhailov, A. S. Simple mechanics of protein machines. J. R. Soc. Interface 16, 20190244 (2019).
20. II. The kinematics of machines. Philos. Trans. R. Soc. Lond. Ser. Contain. Pap. Math. Phys. Character 187, 15-40 (1896).
21. Doyle, L. et al. Rational design of α-helical tandem repeat proteins with closed architectures. Nature 528, 585-588 (2015).
22. Dyer, K. N. et al. High-Throughput SAXS for the Characterization of Biomolecules in Solution: A Practical Approach. in Structural Genomics (ed. Chen, Y. W.) vol. 1091 245-258 (Humana Press, 2014).
23. Hura, G. L. et al. Comprehensive macromolecular conformations mapped by quantitative SAXS analyses. Nat. Methods 10, 453-454 (2013).
24. Grigoryan, G. & DeGrado, W. F. Probing Designability via a Generalized Model of Helical Bundle Geometry. J. Mol. Biol. 405, 1079-1100 (2011).
25. Boyken, S. E. et al. De novo design of protein homo-oligomers with modular hydrogen-bond network-mediated specificity. Science 352, 680-687 (2016).
26. An, L. & Lee, G. R. De Novo Protein Design Using the Blueprint Builder in Rosetta. Curr. Protoc. Protein Sci. 102, (2020).
27. Chaudhury, S., Lyskov, S. & Gray, J. J. PyRosetta: a script-based interface for implementing molecular modeling algorithms using Rosetta. Bioinformatics 26, 689-691 (2010).
28. Maguire, J. B. et al. Perturbing the energy landscape for improved packing during computational protein design. Proteins Struct. Funct. Bioinforma. 89, 436-449 (2021).
29. Punjani, A. & Fleet, D. J. 3D Variability Analysis: Resolving continuous flexibility and discrete heterogeneity from single particle cryo-EM. biorxiv.org/lookup/doi/10.1101/2020.04.08.032466 (2020) doi:10.1101/2020.04.08.032466.
30. Punjani, A., Rubinstein, J. L., Fleet, D. J. & Brubaker, M. A. cryoSPARC: algorithms for rapid unsupervised cryo-EM structure determination. Nat. Methods 14, 290-296 (2017).
31. Sobti, M. et al. Cryo-EM structures provide insight into how E. coli FlFo ATP synthase accommodates symmetry mismatch. Nat. Commun. 11, 2615 (2020).
32. Majewski, D. D. et al. Cryo-EM structure of the homohexameric T3SS ATPase-central stalk complex reveals rotary ATPase-like asymmetry. Nat. Commun. 10, 626 (2019).
33. Clausen, M. V., Hilbers, F. & Poulsen, H. The Structure and Function of the Na,K-ATPase Isoforms in Health and Disease. Front. Physiol. 8, 371 (2017).
34. Woodson, M. et al. A viral genome packaging motor transitions between cyclic and helical symmetry to translocate dsDNA. Sci. Adv. 7, eabc1955 (2021).
35. Deme, J. C. et al. Structures of the stator complex that drives rotation of the bacterial flagellum. Nat. Microbiol. 5, 1553-1564 (2020).
36. Hennell James, R. et al. Structure and mechanism of the proton-driven motor that powers type 9 secretion and gliding motility. Nat. Microbiol. 6, 221-233 (2021).
37. Nakamura, M. et al. Remote control of myosin and kinesin motors using light-activated gearshifting. Nat. Nanotechnol. 9, 693-697 (2014).
38. Crick, F. H. C. The Fourier transform of a coiled-coil. Acta Crystallogr. 6, 685-689 (1953).
39. Fallas, J. A. et al. Computational design of self-assembling cyclic protein homo-oligomers. Nat. Chem. 9, 353-360 (2017).
40. Huang, P.-S. et al. RosettaRemodel: A Generalized Framework for Flexible Backbone Protein Design. PLoS ONE 6, e24109 (2011).
41. Correnti, C. E. et al. Engineering and functionalization of large circular tandem repeat protein nanoparticles. Nat. Struct. Mol. Biol. 27, 342-350 (2020).
42. D. A. Case, H. M. Aktulga, K. Belfon, I. Y. Ben-Shalom, S. R. Brozell, D. S. Cerutti, T. E. Cheatham, III, V. W. D. Cruzeiro, T. A. Darden, R. E. Duke, G. Giambasu, M. K. Gilson, H. Gohlke, A. W. Goetz, R. Harris, S. Izadi, S. A. Izmailov, C. Jin, K. Kasavajhala, M. C. Kaymak, E. King, A. Kovalenko, T. Kurtzman, T. S. Lee, S. LeGrand, P. Li, C. Lin, J. Liu, T. Luchko, R. Luo, M. Machado, V. Man, M. Manathunga, K.M. Merz, Y. Miao, O. Mikhailovskii, G. Monard, H. Nguyen, K. A. O'Hearn, A. Onufriev, F. Pan, S. Pantano, R. Qi, A. Rahnamoun, D. R. Roe, A. Roitberg, C. Sagui, S. Schott-Verdugo, J. Shen, C. L. Simmerling, N. R. Skrynnikov, J. Smith, J. Swails, R. C. Walker, J. Wang, H. Wei, R. M. Wolf, X. Wu, Y. Xue, D. M. York, S. Zhao, and P. A. Kollman. Amber 2018. (University of California).
43. Tian, C. et al. ff19SB: Amino-Acid-Specific Protein Backbone Parameters Trained against Quantum Mechanics Energy Surfaces in Solution. J. Chem. Theory Comput. 16, 528-552 (2020).
44. Roe, D. R. & Brooks, B. R. A protocol for preparing explicitly solvated systems for stable molecular dynamics simulations. J. Chem. Phys. 153, 054123 (2020).
45. Roe, D. R. & Cheatham, T. E. PTRAJ and CPPTRAJ: Software for Processing and Analysis of Molecular Dynamics Trajectory Data. J. Chem. Theory Comput. 9, 3084-3095 (2013).
46. Virtanen, P. et al. SciPy 1.0: fundamental algorithms for scientific computing in Python. Nat. Methods 17, 261-272 (2020).
47. Studier, F. W. Protein production by auto-induction in high-density shaking cultures. Protein Expr. Purif. 41, 207-234 (2005).
48. Förster, S., Apostol, L. & Bras, W. Scatter : software for the analysis of nano- and mesoscale small-angle scattering. J. Appl. Crystallogr. 43, 639-646 (2010).
49. Dyer, K. N. et al. High-Throughput SAXS for the Characterization of Biomolecules in Solution: A Practical Approach. in Structural Genomics (ed. Chen, Y. W.) vol. 1091 245-258 (Humana Press, 2014).
50. Schneidman-Duhovny, D., Hammel, M., Tainer, J. A. & Sali, A. Accurate SAXS Profile Computation and its Assessment by Contrast Variation Experiments. Biophys. J. 1 105, 962-974 (2013).
51. Nannenga, B. L., Iadanza, M. G., Vollmar, B. S. & Gonen, T. Overview of Electron Crystallography of Membrane Proteins: Crystallization and Screening Strategies Using Negative Stain Electron Microscopy. Curr. Protoc. Protein Sci. 72, 17.15.1-17.15.11 (2013).
52. Grant, T., Rohou, A. & Grigorieff, N. cisTEM, user-friendly software for single-particle image processing. eLife 7, e35383 (2018).
53. Punjani, A., Rubinstein, J. L., Fleet, D. J. & Brubaker, M. A. cryoSPARC: algorithms for rapid unsupervised cryo-EM structure determination. Nat. Methods 14, 290-296 (2017).
54. Carragher, B. et al. Leginon: An Automated System for Acquisition of Images from Vitreous Ice Specimens. J. Struct. Biol. 132, 33-45 (2000).
55. Tang, G. et al. EMAN2: An extensible image processing suite for electron microscopy. J. Struct. Biol. 157, 38-46 (2007).
56. Zheng, S. Q. et al. MotionCor2: anisotropic correction of beam-induced motion for improved cryo-electron microscopy. Nat. Methods 14, 331-332 (2017).
57. Rohou, A. & Grigorieff, N. CTFFIND4: Fast and accurate defocus estimation from electron micrographs. J. Struct. Biol. 192, 216-221 (2015).
58. Pettersen, E. F. et al. UCSF Chimera?A visualization system for exploratory research and analysis. J. Comput. Chem. 25, 1605-1612 (2004).
59. Afonine, P. V. et al. Real-space refinement in PHENIX for cryo-EM and crystallography. Acta Crystallogr. Sect. Struct. Biol. 74, 531-544 (2018).
60. VanAernum, Z. L. et al. Rapid online buffer exchange for screening of proteins, protein complexes and cell lysates by native mass spectrometry. Nat. Protoc. 15, 1132-1157 (2020).
61. VanAernum, Z. L. et al. Surface-Induced Dissociation of Noncovalent Protein Complexes in an Extended Mass Range Orbitrap Mass Spectrometer. Anal. Chem. 91, 3611-3618 (2019).

### Materials and Methods

### Computational Design

### Generation of Homooligomeric Dihedral and Cyclic Symmetric Axle Parts:

Approach 1: This approach relies first on the design of short (30 to 50 residues) single alpha helices monomers self-assembling into high aspect ratio dihedral homoligomers, which are then further fused to cyclic wheel shaped homooligomers to yield full axle parts.

Parametric design was used to generate short single α-helices and sample backbone configurations by systematically varying helical parameters using the Crick generating equations(24, 38). As described before, ideal values were used for the supercoil twist (ω0) and helical twist (ω1).

In the case of D3 helical bundles, in order to obtain the helical interdigitated geometry allowing the obtain a packed core without holes after design and therefore assembly of single helices into dihedral symmetry, we sampled two segments with different starting point for the superhelical radii per helix (6 Å and 12 Å), joined by a custom number of linker residues between the two segments, using a custom python script. This parameter range (˜5 Å with bins of 0.5 Å) were chosen based on iteratives cycles of parametric helix generation and Rosetta™ design with metric assessment, and the range of metrics yielding the highest scoring backbone were chosen. The helical phase (Δϕ1) was sampled from 0° to 90° with a step size of 10°. We sampled the offset along the z-axis (Z-offset) from −1.51 Å to 1.51 Å, with a step size of 0.1 Å. The supercoil phases (Δϕ0) were fixed at 0°, and 30° for D3s and D2s, respectively.

Once ideal backbones geometry were generated using this parametric approach, we used the Rosetta™ design protocol to further design side chains identities and rotamers and optimize the interface energy to direct the assembly in dihedral homooligomers. Importantly, this step relied on the use of the Rosetta™ HBnet protocol described previously(25), which allows for extended hydrogen bond networks across monomer subunits therefore ensuring specificity of interaction and symmetric binding mode.

The dihedral building blocks were then rigidly fused to previously designed cyclic homooligomers(39) by designing short rigid helical linkers bridging the two building blocks. The inner helices of the dihedral assemblies obtained (C or N termini depending on design) were then fused by short structured helical fragments using Rosetta™ Remodel(40) while sampling the rotation and distance between Z aligned cyclic homooligomers and dihedral homooligomer. To further stabilize and optimize the generated Cyclic-Dihedral fusion, a second round of Rosetta™ design of the fusion was performed. Method 2: This approach relied on alpha helical extensions of N or C termini of previously designed cyclic homooligomers, in order to direct the assembly of two elongated cyclic homooligomers into high aspect ratio dihedral symmetric axle parts. Rosetta™ SymDofMover was used to set up the symmetry in which the input monomer subunits were aligned along the z axis. Input subunits were first optionally flipped 180 degrees about the z axis to reverse the inputs if necessary, so that the N or C termini to be elongated would point toward each other. Monomer subunits were then translated along the specified z axis and rotated about the z axis according to random Gaussian sampling in order to finely sample helical extension parameters. Following these initial manipulations of the input structures, a symmetric pose was generated using D3, D4, D5, D6 or D8 symmetry definition files. We then applied the Rosetta™ BluePrintBDR mover which allowed us to build helical fragment extension starting at the previously positioned monomers, and spanning the distance between symmetric subunits. Once centroid helical backbones geometries were generated and sampled, we used the Rosetta™ design protocol to further design side chains identities and rotamers and optimize the interface energy to direct the assembly in dihedral homooligomers. Importantly, this step relied on the use of the Rosetta™ HBnet protocol described previously (25), which allows for extended hydrogen bond networks across monomer subunits therefore ensuring specificity of interaction and symmetric binding mode.

### Generation of Cyclic Symmetric Homooligomeric Rotor Parts:

Computationally designed ring shape structures or various symmetries (C1, C3, C4)' were either collected from previously published work (21,41), or designed from heterodimers and

DHRs in symmetry mode (C3, C5) using protocols previously described(/2). 9x, 12x and 24x toroids were used in C1 symmetric versions or cut into 3 or 4 to produce C3 or C4 symmetric homooligomers. All designs were then computationally augmented by systematic symmetric fusion of DHR repeats proteins using the HFuse protocol, and the surrounding fusion interface of the fusion was further redesigned using Rosetta™ design protocols to optimize the assembly energy.

Generation of Two Component Rotary Machine Models from Symmetric Axle and Rotor Parts:

The goal of the computational docking procedure between axle and rotor machine parts was to exhaustively sample the rotational conformational space within some specified resolution and meaningful interface quality, all possible ways to assemble a full rotary machine complex from the two libraries of previously designed axle and rotor parts.

We started by enumerating all possible rotary machine assemblies by inspecting shapes and dimensions of available parts and identifying assemblies that would not produce any steric clashes. We then proceeded to computational docking of parts using a two-dimensional rigid body docking space to allow contact between the axle and rotor (one rotation and one translation along the Z axis). We sampled 180° rotation for C2s, 120° for C3s, 90° for C4s, 72° for C5s, and we sampled the whole span on available translation along the axis that would not generate clashes between backbones, with a 1° and 1 Å step, respectively. For each sampled dock, the resulting heteromultimeric interface was designed either using Rosetta™ design and HBnet to obtain tightly packed, specific interfaces with extended hydrogen bond networks, and in some cases by constraining the residue identities of the axle (DEHQTNSY) and ring (KRHQTNSY) to obtain complementary charges allowing loose non specific interactions. Since some of the resulting assemblies have intrinsic symmetry mismatch between the axle and rotor (e.g. D8 axle and C4 ring), we used a quasi-symmetric design methodology, relying on the Rosetta™ StoreQuasiSymmetricTaskMover, which creates a stored task that links selected interface residues. The residues remain identical in identity when the interface is designed, but their rotamers are packed differently, which allows identical residues in symmetric subunits to satisfy multiple interfaces at the same time.

In order to kinetically trap rings onto the axle, we further generated a disulfided version of homooligomers by placing cysteine at the interface between asymmetric units. This was achieved using a PyRosetta™ based stapling method that allows to identify pairs of residues that can accommodate disulfides given the 3D structure of a protein.

### Interface Disulfide Stapling

This protocol was developed to quickly identify pairs of residues that can accommodate disulfides given the 3D structure of a protein. 30,000 native disulfide structures were procured from the PDB, and the relative positions of the backbone atoms (N, CA, C) were calculated, hashed, and stored into a database. A candidate protein structure can then be searched for residue pairs at all relative positions of backbone atoms that can accommodate disulfides according to native geometries.

### Molecular Dynamics Simulations

Rosetta™ models of D3-C3 and D3-C5 with truncated ring DHR arms (to minimize the total number of atoms to simulate) were used as the starting coordinates for the simulations. The rotor rings of D3-C3 and D3-C5 were rotated at 10 and 12 degree intervals, respectively. Each model was solvated in an octahedral periodic box of OPC water and 70 mM NaCl using AmberTools18 (42). In total, each system consisted of approximately 590,000 atoms. Simulations were run at constant pressure (1 bar) and temperature (298 K) using the Monte Carlo barostat, the Langevin thermostat and the ff19SB forcefield(43). Using the CUDA enabled version of Amber18, four parallel simulations for each rotated model were equilibrated using the AmberMDprep protocol(44). Once equilibrated, the simulations were run at 2 fs timestep for a total of 40 ns each, yielding an aggregate simulation time of 1920 ns for D3-C3 and 960 ns for D3-05. To allow exploration of the rotors' degrees of freedom from the initial configurations, the first 20 ns of each simulation was discarded and the final 20 ns was used in later analysis. To investigate the movement of the rings around their respective axles, 200 ps snapshots of the simulations were aligned to the initial axle coordinates by rmsd. Number density maps of the backbone atoms were calculated using the VolMap command in AmberTool's cpptraj (45). These maps were contoured to 0.001 as shown in fig. S15. To calculate the axle drift with respect to ring rotation, the backbone center of mass of the rings was calculated for all aligned snapshots. The snapshots were binned according to the ring rotation in 24 degree intervals and then averaged as shown in fig. S15. To calculate ring tilt, the centers of mass of each ring subunit was calculated, then a plane was fit through these points using the least squares optimizer in SciPy (46). The angle between this plane and the long axis of the axle was taken as the tilt, and this was averaged over rotation as described for the axial drift. The Mean Square Displacement for the DOFs was computed as MSD=average(r(t)−(0))\hat{ }2.

Buffer and media recipe for protein expression

TBM-5052: 1.5% [wt/vol] tryptone, 2.5% [wt/vol] yeast extract, 0.5% [wt/vol] glycerol, 0.05% [wt/vol] D-glucose, 0.2% [wt/vol] D-lactose, 25 mM Na2HPO4, 25 mM KH2PO4, 50 mM NH4Cl,5 mM Na2SO4, 2 mM MgSO4, 10 μM FeCl3, 4 μM CaC12, 2 μM MnC12, 2 μM ZnSO4, 400 nM CoC12, 400 nM NiCl2, 400 nM CuCl2, 400 nM Na2MoO4, 400 nM Na2SeO3, 400 nM H3BO3

Lysis buffer: 25 mM Tris, 25 mM NaCl, 20 mM Imidazole, pH 8.0 at room temperature

Wash buffer: 25 mM Tris, 25mM NaCl, 20 mM Imidazole, pH 8.0 at room temperature

Elution buffer: 25 mM Tris, 25 mM NaCl, 200 mM Imidazole, 50mM EDTA, pH 8.0 at room temperature

TBS buffer: 25 mM Tris pH 8.0, 25 mM NaCl

Construction of synthetic genes

Prior to transformation and expression in E coli hosts, synthetic genes were ordered either from Integrated DNA Technologies (Coralville, IA) or Genscript Inc. (Piscataway, N.J., USA) and cloned in pET29b+e. coli expression vector between the NdeI and Xhof sites. For bicistronic constructs used for screening the in cellulo assembly of axle and rotors, a synthetic bicistron containing both axle and rotor genes were synthesised and cloned at once in the Ndel/Xhof site, with a termination and strong ribosomal binding site sequence between the genes. For most synthetic gene constructs, a C or N ter hexahistidine tag was added in frame after a short GS linker. A stop codon was introduced at the 3′ end of the protein coding sequence to prevent expression of the C-terminal hexahistidine tag in the vector.

### Protein Expression

Plasmids were transformed into chemically competent E. coli expression strain BL21(DE3*) (New England Biolabs) for protein expression. Following transformation and overnight growth on Luria-Bertani agar Kanamycin plates 100 ug/ml, single colonies were picked and directly transferred into 2×50 ml TBM-5052 medium containing 150 μg/mL Kanamycin and incubated with shaking at 225 rpm for 24 hours at 37° C. following the autoinduction method (47). After 24 hours of incubation, the temperature was dropped for an overnight incubation at 20° C. before harvesting the cells via centrifugation at 4500 G for 20 minutes at 4° C.

### Affinity Purification

The cell pellets were resuspended in 30 ml lysis buffer, followed by cell lysis via sonication at 85% power for 2.5 minutes (10 sec on/10 sec off) while keeping the cell suspension at 4° C. Lysates were clarified by centrifugation at 4° C. and 18000 G for 45 minutes and applied to columns containing Ni-NTA (Qiagen) resin pre-equilibrated with lysis buffer. The columns were washed 3 times with 10 column volumes (CV) of wash buffer, followed by 15 ml of elution buffer for protein elution.

### Size-Exclusion Chromatography (SEC)

Protein elutions were further concentrated in 15mL 3K protein concentrators (Millipore Sigma) to a volume of 500uL and the buffer exchanged for TBS buffer. The resulting protein solutions were purified by SEC using a Superdex™ 6 10/300 GL increase column (GE Healthcare) or a Superdex™ 200 10/300 GL increase column in TBS buffer. SEC elution fractions corresponding to the designs theoretical elution volumes were concentrated in TBS prior to further biochemical analysis. The theoretical SEC elution volumes were computed using the following calibrated equations: V_{S200}=−1.89 log(<mass of design in kDa>)+21.9 ; and V_{S6}32 −1.33 log(<mass of design in kDa>)+21.9.

### D3-C3 and D3-C5 Assembly Process

D3 axles and C3 or C5 rings were purified as previously described. Axle and ring were then mixed in TBS solution with 25mM TCEP following a 1:1 stoichiometry, after which the pH is dropped to 3.0 by dialysis in citrate buffer with TCEP. The protein samples were then heated for an hour at 65C, and then allowed to cool back down to room temperature on a bench. The protein samples were then dialysed overnight in TBS buffer and further SEC purified.

### Small Angle X-ray Scattering (SAXS)

Protein samples were purified by SEC in 25 mM Tris pH 8.0, 25 mM NaCl and 1% glycerol; elution fractions corresponding to the protein were further concentrated using 3K protein concentrators (Millipore Sigma) and the flow-through was used as blank for buffer subtraction. SAXS Scattering measurements were performed at the SIBYLS 12.3.1 beamline at the Advanced Light Source. The sample-to-detector distance was 1.5 m, and the X-ray wavelength (X) was 1.27 Å, corresponding to a scattering vector q (q=4πsin θ/λ, where 2θ is the scattering angle) range of 0.01 to 0.3 Å-1. Å series of exposures were taken of each well, in equal sub-second time slices: 0.3-s exposures for 10 s resulting in 32 frames per sample. For each sample, data were collected for two different concentrations to test for concentration-dependent effects; ‘low’ concentration samples corresponded to 1 mg/ml and ‘high’ concentration samples to 5 mg/ml. Collected data were processed using the SAXS FrameSlice™ online server and analysed using the ScÅtter software package(23). The FoXS™ software (Sali Lab) was used to compare experimental scattering profiles to design models and assess quality of fit(48-50).

### Electron Microscopy

### Negative Stain Electron Microscopy:

SEC fractions corresponding to the designs were concentrated in TBS prior to negative stain EM screening. Samples were then immediately diluted 5 to 150 times in TBS buffer (tris 25mM, NaCl 25mM) depending on the concentration of the samples. A final volume of 5 μL was applied on negatively glow discharged, carbon-coated 400-mesh copper grids (01844-F, TedPella,Inc.), then washed with Milli-Q Water and stained using 0.75% uranyl formate as previously described (51). Air-dried grids were then imaged on either a FEI Talos L120C TEM (FEI Thermo Scientific, Hillsboro, OR) equipped with a 4K×4K Gatan OneView™ camera at a magnification of 57,000× and pixel size of 2.51 Å. Micrographs collection was automated using EPU software (FEI Thermo Scientific, Hillsboro, OR) and were imported into CisTEM software (52) or cryoSPARC software (53). CTF estimation was done with CTFFIND4 and a circular blob picker was used to select particles which were then subjected to 2D classification. Ab initio reconstruction and homogeneous refinement in Cn symmetry were used to generate 3D electron density maps.

### CryoEM Sample Preparation and DataCollection:

CryoEM grids were prepared by diluting protein samples with TBS 1 to 10 times immediately before applying 3.5 μL to glow-discharged 400 mesh, C-flat, 2 micron holes, 2 micron spacing, CF-2/2-4C (CF-224C-100) (Electron Microscopy Sciences, Hatfield, PA) cryoEM grids. For some samples, multiple blots were applied in order to obtain the best particle density. All grids were blotted using a blot force of 0 and 5 second blot time at 100% humidity and 4° C. and plunge-frozen in liquid ethane using a Vitrobot™ Mark IV (FEI Thermo Scientific, Hillsboro, OR). All cryoEM grids were screened on a Glacios transmission electron microscope (FEI Thermo Scientific, Hillsboro, OR) operated at 200 kV and equipped with a Gatan K2 Summit direct detector. Automated glacios data collection was carried out using Leginon (54) at a nominal magnification of 36,000× (1.16 Å/pixel). Movies were acquired in counting mode fractionated in 50 frames of 200 ms at 8.5 e-/pixel/sec for a total dose of ˜65e-/Å². High resolution data was collected on a Titan Krios™ (FEIco.) operating at 300 kV, with a Quantum GIF energy filter (GatanInc.) operating in zero-loss mode with a 20eV slit width, and a K-2 Summit Direct Detect™ camera. Movies were acquired using Leginon in super-resolution mode at 130,000× (pixel size 0.525 Å/pixel) with 50 frames at an exposure rate of 2.5 e-/pixel/sec for a total dose of ˜90e/Å². Details of dataset processing for each design are illustrated in Table S1 and Figure S3, S5, S6, S12 and S13. Theoretical 2D projections were generated using CryoSparc software's “create template” function from an input volume generated with EMAN2 (55).

### CryoEM Data Processing:

Multiple datasets were collected for each design and combined early on during processing. See table 1 and processing flowcharts for details. Briefly, images were manually curated to remove poor quality acquisitions such as bad ice or large regions of carbon. Dose-weighting and image alignment of all 50 frames was carried out using MotionCor2 (56) with 5×5 patch or with cryosparc v2 patch alignment tool with default parameters. Super-resolution krios data was binned 2X during alignment. Initial CTF parameters were estimated using CTFfind4 (57). Particle picking was done with a gaussian blob picker and in some cases followed by a template picker. Particles were extensively classified in 2D to remove junk particles and designs which may not have been intact or were damaged, yielding in some cases relatively few particles. This may also be due to the low mass of the designed proteins which did not align well. In addition, the expected motion of the rotors may have introduced further heterogeneity, limiting classification efforts. Starting models for all designs were always obtained ab initio, despite clear evidence of the expected design in 2D. In 3D classification and refinement we were able to resolve either axle or ring, and in one case both together (D8-C4), suggesting rotor movement. FSC 0.143 curves were generated by exporting half maps to relion for post-process. Local resolution estimates were generated in relion and displayed onto the locally filtered map outputs using Chimera (58). For density modification in Phenix (59), we used as input the exported half maps from cryosparc with default params at 100 bins and local filtering with a factor of 5. FSC curves were plotted using the Phenix density modification Fref 0.5 output along with the relion FSC estimates. Directional FSC calculated using remote 3DF SC processing tool. 3D Variability analysis (3DVA) of the D8-C4 design was done in cryosparc v2 following expanded particles in D4 symmetry of the final reconstructions with a mask around both rings and the axel. We used default settings of simple cluster mode and 10 frame output with a 10 Å lowpass filter for assessing variability. First and last frames of the second trajectory component were used as input for downstream refinement of distinct structures. Resulting maps were then low-pass filtered to 15 Å for clarity. For D3-C5, 3DVA was carried out after D3 symmetry was expanded and variability was processed and filtered at 5 Å for display.

### Biolayer Interferometry

Biolayer interferometry experiments were performed on an OctetRED96 BLI system (ForteBio, Menlo Park, CA). Enzymatic protein biotinylation was performed on SEC purified Avi-tagged proteins prior to the assay. The BirA500 (Avidity, LLC) biotinylation kit was used to biotinylate protein from the IMAC elution according to the manufacturer protocol. Reactions were incubated at 4C overnight and purified using size exclusion chromatography on a Superdex™ 6 10/300 Increase GL (GE Healthcare) in TBS buffer (25 mM Tris pH 8.0, 25 mM NaCl). Streptavidin coated biosensors were equilibrated for 10 minutes in Octet buffer (10 mM HEPES pH 7.4, 25 mM NaCl, 3 mM EDTA, 0.05% Surfactant P20) supplemented with 1 mg/ml Bovine Serum Albumin (SigmaAldrich). Enzymatically biotinylated axle components were immobilized onto the biosensors by dipping the biosensors into a solution with 10-50 nM protein for 200-500s. This was followed by dipping in fresh octet buffer to establish a baseline. Titration experiments were performed at 25 ° C. while rotating at 1,000 r.p.m. Association of rings rotor components with axle immobilized on the tips was allowed by dipping biosensors in solutions containing designed protein diluted in octet buffer followed by dissociation by dipping the biosensors into fresh buffer solution in order to monitor the dissociation kinetics.

### Native Mass Spectrometry

The oligomeric state of in vivo assembled rotors was analyzed by online buffer exchange MS(60) using a Vanquish UHPLC coupled to a Q Exactive™ Ultra-High Mass Range (UHMR) mass spectrometer (Thermo Fisher Scientific) modified to allow for surface-induced dissociation (SID) similar to that previously described (61). 1 μL of 25 μM protein in TBS buffer were injected and online buffer exchanged into 200 mM ammonium acetate, pH 6.8 by a self-packed buffer exchange column (P6 polyacrylamide gel, Bio-Rad Laboratories) at a flow rate of 100 μL per min. A heated electrospray ionization (HEST) source with a spray voltage of 4 kV was used for ionization. Mass spectra were recorded for 1000-20000 m/z at 3125 resolution as defined at 400 m/z. The injection time was set to 200 ms. Voltages applied to the transfer optics were optimized to allow for ion transmission while minimizing unintentional ion activation, and a higher-energy collisional dissociation of 5 V was applied. Mass spectra were deconvolved using UniDec V4.2.2 22. Deconvolution settings included mass sampling every 10 Da, smooth charge states distributions, automatic peak width tool, point smooth width of 1 or 10, and beta of 50.

We claim:

1. A polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-15 and 17-51, not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity.

2. The polypeptide of claim 1, wherein any amino acid substitutions at interface residues (single underlined residues) are conservative amino acid substitutions.

3. The polypeptide of claim 1, wherein any amino acid substitutions at structural residues (bold font residues) are conservative amino acid substitutions.

4. The polypeptide of claim 1, wherein any amino acid substitutions at residues needed for binding to small molecule (residues within squiggly brackets) are conservative amino acid substitutions.

5. The polypeptide of claim 1, wherein one or more loop regions are substituted or added to with any peptide domain deemed suitable for an intended use: domains that can be modified by enzymatic activity (i.e. phosphorylation), small molecule or protein binding domains, or catalytic domains.

6. The polypeptide of claim 1, wherein interface residues are not substituted. 7 The polypeptide of claim 1, wherein structural residues are not substituted.

8. The polypeptide of claim 1, wherein residues needed for binding to small molecule are not substituted.

9. The polypeptide of claim 1, wherein optional amino acid residues are absent and are not considered when determining percent identity.

10. A kit or machine assembly, comprising an axle and ring pair comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of one or more axle and ring pair are selected from the group consisting of the following pairs (A)-(J), not including any functional domains added fused to the polypeptides (whether N-terminal, C-terminal, or internal), and wherein the 1, 2, 3, 4, or 5 N-terminal and/or C-terminal amino acid residues may be present or absent and when absent are not considered in determining the percent identity.
(A) SEQ ID NO:5 and SEQ ID NO:6;
(B) SEQ ID NO:7 and SEQ ID NO:8;
(C) SEQ ID NO:9 and SEQ ID NO:10;
(D) SEQ ID NO:11 and SEQ ID NO:12;
(E) SEQ ID NO:13 and SEQ ID NO:14;
(F) SEQ ID NO:15 and SEQ ID NO:17;
(G) SEQ ID NO:18 and SEQ ID NO:19;
(H) SEQ ID NO:20 and SEQ ID NO:21;
(I) SEQ ID NO:22 and SEQ ID NO:23; and/or
(J) SEQ ID NO:24 and SEQ ID NO:25.

11. The kit or machine assembly of claim 10, wherein any amino acid substitutions at interface residues (single underlined residues) are conservative amino acid substitutions.

12. The kit or machine assembly of claim 10, wherein any amino acid substitutions at structural residues (bold font residues) are conservative amino acid substitutions.

13. The kit or machine assembly of claim 10, wherein any amino acid substitutions at residues needed for binding to small molecule (residues within squiggly brackets) are conservative amino acid substitutions.

14. The kit or machine assembly of claim 10, wherein one or more loop regions are substituted or added to with any peptide domain deemed suitable for an intended use. The kit or machine assembly of claim 10, wherein interface residues, structural residues, and/or residues needed for binding to small molecul are not substituted.

16. The kit or machine assembly of claim 10, wherein optional amino acid residues are absent and are not considered when determining percent identity.

17. A nucleic acid encoding the polypeptide of claim 1.

18. An expression vector comprising the nucleic acid of claim 17 operatively linked to a suitable control sequence.

19. A host cell comprising the expression vector of claim 18.

20. A method for using the kit or machine of claim 10 for any suitable use as disclosed herein.
